SEQUENCE LISTING (27 sequences)

SEQ ID NO: 1 - DNA, 3351 bp, Tobacco mosaic virus
  CDS (1)..(3348)
atggcatacacacagacagctaccacatcagctttgctggacactgtccgaggaaacaactccttggtcaatgatctagcaaagcgtcgtctttacgacacagcggttgaagagtttaacgctcgtgaccgcaggcccaaagtgaacttttcaaaagtaataagcgaggagcagacgcttattgctacccgggcgtatccagaattccaaattacattttataacacgcaaaatgccgtgcattcgcttgcaggtggattgcgatctttagaactggaatatctgatgatgcaaattccctacggatcattgacttatgacataggcgggaattttgcatcgcatctgttcaagggacgagcatatgtacactgctgcatgcccaacctggacgttcgagacatcatgcggcatgaaggccagaaagacagtattgaactatacctttctaggctagagagagggggaaaaacagtccccaacttccaaaaggaagcatttgacagatacgcagaaattcctgaagacgctgtctgtcacaatactttccagacatgcgaacatcagccgatgcaacaatcaggcagagtgtatgccattgcgctacacagcatatatgacatacccgctgatgagttcggggcagcactcttgaggaaaaatgtccatacgtgctatgccgctttccacttctctgagaacctgcttcttgaagattcatacgtcaatctggacgaaatcaacgcgtgtttttcgcgcgatggagacaagttgaccttttcttttgcatcagagagtactcttaattactgtcatagttattctaatattcttaagtatgtgtgcaaaacttacttcccggcctctaatagagaggtttacatgaaggagtttttagtcaccagggttaatacctggttttgtaagttttctagaatagatacttttcttttgtacaaaggtgtggcccataaaggtgtagatagtgagcagttttatactgcaatggaagacgcatggcattacaaaaagactcttgcaatgtgcaacagcgagagaatcctccttgaggattcatcaacagtcaattactggtttcccgaaatgagggatatggtcatcgtaccattattcgacatttctttggagactagtaagaggacgcgcaaggaagtcttagtgtccaaggatttcgtgtttacagtgcttaaccacattcgaacataccaggcaaaagctcttacatacgtaaatgttttgtccttcgtcgaatcgattcgatcgagggtaatcattaacggtgtgacagcgaggtccgaatgggatgtggacaaatctttgttacaatccttgtccatgacgttttacctgcatactaagcttgccgttctaaaggatgacttactgattagcaagtttagtctcggttcgaaaacggtgtgccagcatgtgtgggatgagatttcactggcgtttgggaacgcatttccctccgtgaaagagaggctcttgaacaggaaacttatcagagtggcaggcgacgcactagagatcagggtgcctgatctatatgtgaccttccacgaccgattagtgactgagtacaaggcctctgtggacatgcctgcgcttgacattaggaagaagatggaagaaacggaagtgatgtacaatgcactttcagagttatcggtgttaagggagtctgacaaattcgatgttgatgttttttcccagatgtgccaatctttggaagttgacgcaatgacggcagcgaaggttatagtcgcggtcatgagcaataagagcggtctgactctcacatttgaacgacctactgaggcgaatgttgcgctagctttacaggatcaagaaaaggcttcagaaggtgctttggtagttacctcaagagaagttgaagaaccgtccatgaagggttcgatggccagaggagagttacaattagctggtcttgctggagatcatccggagtcgtcctattctaggaacgaggagatagagtctttagagcagtttcatatggcaacggcagattcgttaattcgtaagcagatgagctcgattgtgtacacgggtccgattaaagttcagcaaatgaaaaactttatcgatagcctggtagcatcactatctgctgcggtgtcgaatctcgtcaagatcctcaaagatacagctgctattgaccttgaaacccgtcaaaagtttggagtcttggatgttacatctaggaagtggttaattaaaccaacggccaagagtcatgcatggggtgttgttgaaacccacgcgaggaagtatcatgtggcgcttctggaatatgatgagcagggtgtggtgacatgcgatgattggagaagagtagctgtcagctctgagtctgttgtttattccgacatggcgaaactcagaactctgcgcagactgcttcgaaacggagaaccgcatgtcagtagcgcaaaggttgttcttgtggacggagttccgggctgtggaaaaaccaaagaaattctttccagggttaattttgatgaagatctaattttagtacctgggaagcaagctgctgaaatgatcagaagacgtgcgaattcctcagggattattgtggccacgaaggacaacgttaaaaccgttgattctttcatgatgaattttgggaaaagcacacgctgtcagttcaagaggttattcattgatgaagggttgatgttgcatactggttgtgttaattttcttgtggcgatgtcattgtgcgaaattgcatatgtttacggagacacacagcagattccatacatcaatagagtttcaggattcccgtaccccgcccattttgccaaattggaagttgacgaggtggagacacgcagaactactctccgttgtccagccgatgtcacacattatctgaacaggagatatgagggctttgtcatgagcacttcttcggttaaaaagtctgtttcgcaggagatggtcggcggagccgccgtgatcaatccgatctcaaaacccttgcatggcaagatcctgacttttacccaatcggataaagaagctctgctttcaagagggtattcagatgttcacactgtgcatgaagtgcaaggcgagacatactctgatgtttcactagttaggctaacccctacaccagtctccatcattgcaggagacagcccgcatgttttggtcgcattgtcaaggcacacctgttcgctcaagtactacactgttgttatggatcctttagttagtatcattagagatctagagaaacttagctcgtacttgttagatatgtataaggtcgatgcaggaacacaatag

SEQ ID NO: 2 - protein, 1116 aa, Tobacco mosaic virus
MAYTQTATTSALLDTVRGNNSLVNDLAKRRLYDTAVEEFNARDRRPKVNFSKVISEEQTLIATRAYPEFQITFYNTQNAVHSLAGGLRSLELEYLMMQIPYGSLTYDIGGNFASHLFKGRAYVHCCMPNLDVRDIMRHEGQKDSIELYLSRLERGGKTVPNFQKEAFDRYAEIPEDAVCHNTFQTCEHQPMQQSGRVYAIALHSIYDIPADEFGAALLRKNVHTCYAAFHFSENLLLEDSYVNLDEINACFSRDGDKLTFSFASESTLNYCHSYSNILKYVCKTYFPASNREVYMKEFLVTRVNTWFCKFSRIDTFLLYKGVAHKGVDSEQFYTAMEDAWHYKKTLAMCNSERILLEDSSTVNYWFPEMRDMVIVPLFDISLETSKRTRKEVLVSKDFVFTVLNHIRTYQAKALTYVNVLSFVESIRSRVIINGVTARSEWDVDKSLLQSLSMTFYLHTKLAVLKDDLLISKFSLGSKTVCQHVWDEISLAFGNAFPSVKERLLNRKLIRVAGDALEIRVPDLYVTFHDRLVTEYKASVDMPALDIRKKMEETEVMYNALSELSVLRESDKFDVDVFSQMCQSLEVDAMTAAKVIVAVMSNKSGLTLTFERPTEANVALALQDQEKASEGALVVTSREVEEPSMKGSMARGELQLAGLAGDHPESSYSRNEEIESLEQFHMATADSLIRKQMSSIVYTGPIKVQQMKNFIDSLVASLSAAVSNLVKILKDTAAIDLETRQKFGVLDVTSRKWLIKPTAKSHAWGVVETHARKYHVALLEYDEQGVVTCDDWRRVAVSSESVVYSDMAKLRTLRRLLRNGEPHVSSAKVVLVDGVPGCGKTKEILSRVNFDEDLILVPGKQAAEMIRRRANSSGIIVATKDNVKTVDSFMMNFGKSTRCQFKRLFIDEGLMLHTGCVNFLVAMSLCEIAYVYGDTQQIPYINRVSGFPYPAHFAKLEVDEVETRRTTLRCPADVTHYLNRRYEGFVMSTSSVKKSVSQEMVGGAAVINPISKPLHGKILTFTQSDKEALLSRGYSDVHTVHEVQGETYSDVSLVRLTPTPVSIIAGDSPHVLVALSRHTCSLKYYTVVMDPLVSIIRDLEKLSSYLLDMYKVDAGTQ

SEQ ID NO: 3 - DNA, 4834 bp, Tobacco mosaic virus
  gene (1)..(4831)
atggcatacacacagacagctaccacatcagctttgctggacactgtccgaggaaacaactccttggtcaatgatctagcaaagcgtcgtctttacgacacagcggttgaagagtttaacgctcgtgaccgcaggcccaaagtgaacttttcaaaagtaataagcgaggagcagacgcttattgctacccgggcgtatccagaattccaaattacattttataacacgcaaaatgccgtgcattcgcttgcaggtggattgcgatctttagaactggaatatctgatgatgcaaattccctacggatcattgacttatgacataggcgggaattttgcatcgcatctgttcaagggacgagcatatgtacactgctgcatgcccaacctggacgttcgagacatcatgcggcatgaaggccagaaagacagtattgaactatacctttctaggctagagagagggggaaaaacagtccccaacttccaaaaggaagcatttgacagatacgcagaaattcctgaagacgctgtctgtcacaatactttccagacatgcgaacatcagccgatgcaacaatcaggcagagtgtatgccattgcgctacacagcatatatgacatacccgctgatgagttcggggcagcactcttgaggaaaaatgtccatacgtgctatgccgctttccacttctctgagaacctgcttcttgaagattcatacgtcaatctggacgaaatcaacgcgtgtttttcgcgcgatggagacaagttgaccttttcttttgcatcagagagtactcttaattactgtcatagttattctaatattcttaagtatgtgtgcaaaacttacttcccggcctctaatagagaggtttacatgaaggagtttttagtcaccagggttaatacctggttttgtaagttttctagaatagatacttttcttttgtacaaaggtgtggcccataaaggtgtagatagtgagcagttttatactgcaatggaagacgcatggcattacaaaaagactcttgcaatgtgcaacagcgagagaatcctccttgaggattcatcaacagtcaattactggtttcccgaaatgagggatatggtcatcgtaccattattcgacatttctttggagactagtaagaggacgcgcaaggaagtcttagtgtccaaggatttcgtgtttacagtgcttaaccacattcgaacataccaggcaaaagctcttacatacgtaaatgttttgtccttcgtcgaatcgattcgatcgagggtaatcattaacggtgtgacagcgaggtccgaatgggatgtggacaaatctttgttacaatccttgtccatgacgttttacctgcatactaagcttgccgttctaaaggatgacttactgattagcaagtttagtctcggttcgaaaacggtgtgccagcatgtgtgggatgagatttcactggcgtttgggaacgcatttccctccgtgaaagagaggctcttgaacaggaaacttatcagagtggcaggcgacgcactagagatcagggtgcctgatctatatgtgaccttccacgaccgattagtgactgagtacaaggcctctgtggacatgcctgcgcttgacattaggaagaagatggaagaaacggaagtgatgtacaatgcactttcagagttatcggtgttaagggagtctgacaaattcgatgttgatgttttttcccagatgtgccaatctttggaagttgacgcaatgacggcagcgaaggttatagtcgcggtcatgagcaataagagcggtctgactctcacatttgaacgacctactgaggcgaatgttgcgctagctttacaggatcaagaaaaggcttcagaaggtgctttggtagttacctcaagagaagttgaagaaccgtccatgaagggttcgatggccagaggagagttacaattagctggtcttgctggagatcatccggagtcgtcctattctaggaacgaggagatagagtctttagagcagtttcatatggcaacggcagattcgttaattcgtaagcagatgagctcgattgtgtacacgggtccgattaaagttcagcaaatgaaaaactttatcgatagcctggtagcatcactatctgctgcggtgtcgaatctcgtcaagatcctcaaagatacagctgctattgaccttgaaacccgtcaaaagtttggagtcttggatgttacatctaggaagtggttaattaaaccaacggccaagagtcatgcatggggtgttgttgaaacccacgcgaggaagtatcatgtggcgcttctggaatatgatgagcagggtgtggtgacatgcgatgattggagaagagtagctgtcagctctgagtctgttgtttattccgacatggcgaaactcagaactctgcgcagactgcttcgaaacggagaaccgcatgtcagtagcgcaaaggttgttcttgtggacggagttccgggctgtggaaaaaccaaagaaattctttccagggttaattttgatgaagatctaattttagtacctgggaagcaagctgctgaaatgatcagaagacgtgcgaattcctcagggattattgtggccacgaaggacaacgttaaaaccgttgattctttcatgatgaattttgggaaaagcacacgctgtcagttcaagaggttattcattgatgaagggttgatgttgcatactggttgtgttaattttcttgtggcgatgtcattgtgcgaaattgcatatgtttacggagacacacagcagattccatacatcaatagagtttcaggattcccgtaccccgcccattttgccaaattggaagttgacgaggtggagacacgcagaactactctccgttgtccagccgatgtcacacattatctgaacaggagatatgagggctttgtcatgagcacttcttcggttaaaaagtctgtttcgcaggagatggtcggcggagccgccgtgatcaatccgatctcaaaacccttgcatggcaagatcctgacttttacccaatcggataaagaagctctgctttcaagagggtattcagatgttcacactgtgcatgaagtgcaaggcgagacatactctgatgtttcactagttaggctaacccctacaccagtctccatcattgcaggagacagcccgcatgttttggtcgcattgtcaaggcacacctgttcgctcaagtactacactgttgttatggatcctttagttagtatcattagagatctagagaaacttagctcgtacttgttagatatgtataaggtcgatgcaggaacacaatagcaattacagattgactcggtgttcaaaggttccaatctttttgtggcagcgccaaagactggtgatatttctgatatgcagttttactatgataagtgtctcccaggcaacagcaccatgatgaataattttgatgctgttaccatgaggttgactgacatttcattgaatgtcaaagattgcatattggatatgtctaagtctgttgctgcgcctaaggatcaaatcaaaccactaatacctatggtacgaacggcggcagaaatgccacgccagactggactattggaaaatttagtggcgatgattaaaaggaactttaacgcacccgagttgtctggcatcattgatattgaaaatactgcatctttagttgtagataagtttttcgatagttatttgcttaaagaaaaaagaaaaccaaataaaaatgtttctttgttcagtagagagtctctcaatagatggttagaaaagcaggaacaggtaacaataggccagctcgcagattttgattttgtagatttgccagcagttgatcagtacagacacatgatcaaagcacaacccaagcaaaaattggacacttcaatccaaacggagtacccggctttgcagacgattgtgtaccattcgaaaaagatcaatgcaatatttggcccgttgtttagtgagcttactaggcaattactggacagtgttgattcgagcagatttttgtttttcacaagaaagacaccagcgcagattgaggatttcttcggagatctcgacagtcatgtgccgatggatgtcttggagctggatatatcaaaatacgacaaatctcagaatgaattccactgtgcagtagaatacgagatttggcgaagattgggttttgaagacttcttgggagaagtttggaaacaagggcatagaaagaccaccctcaaggattataccgcaggtatcaaaacttgcatctggtatcaaagaaagagtggggacgtcacgacattcattggaaacactgtgatcattgctgcatgtttggcctcgatgcttccgatggagaaaataatcaaaggagccttttgtggtgacgatagtctgctgtacttcccaaagggttgtgagtttccggatgtgcaacactccgcgaatcttatgtggaattttgaagcaaaactgtttaaaaaacagtatggatacttttgcggaagatatgtaatacatcacgacagaggatgcattgtgtattacgatcccctaaagttgatctcgaaacttggcgctaaacacatcaaggattgggaacacttggaggagttcagaaggtctctttgtgatgttgctgtttcgttgaacaattgtgcgtattatacacagttggacgacgctgtatgggaggttcataagaccgcccctccaggttcgtttgtttataaaagtctggtgaagtatttgtctgataaagttctttttagaagtttgtttatag

SEQ ID NO: 4 - protein, 1616 aa, Tobacco mosaic virus
  misc_feature (1117)..(1117): Xaa is unknown
MAYTQTATTSALLDTVRGNNSLVNDLAKRRLYDTAVEEFNARDRRPKVNFSKVISEEQTLIATRAYPEFQITFYNTQNAVHSLAGGLRSLELEYLMMQIPYGSLTYDIGGNFASHLFKGRAYVHCCMPNLDVRDIMRHEGQKDSIELYLSRLERGGKTVPNFQKEAFDRYAEIPEDAVCHNTFQTCEHQPMQQSGRVYAIALHSIYDIPADEFGAALLRKNVHTCYAAFHFSENLLLEDSYVNLDEINACFSRDGDKLTFSFASESTLNYCHSYSNILKYVCKTYFPASNREVYMKEFLVTRVNTWFCKFSRIDTFLLYKGVAHKGVDSEQFYTAMEDAWHYKKTLAMCNSERILLEDSSTVNYWFPEMRDMVIVPLFDISLETSKRTRKEVLVSKDFVFTVLNHIRTYQAKALTYVNVLSFVESIRSRVIINGVTARSEWDVDKSLLQSLSMTFYLHTKLAVLKDDLLISKFSLGSKTVCQHVWDEISLAFGNAFPSVKERLLNRKLIRVAGDALEIRVPDLYVTFHDRLVTEYKASVDMPALDIRKKMEETEVMYNALSELSVLRESDKFDVDVFSQMCQSLEVDAMTAAKVIVAVMSNKSGLTLTFERPTEANVALALQDQEKASEGALVVTSREVEEPSMKGSMARGELQLAGLAGDHPESSYSRNEEIESLEQFHMATADSLIRKQMSSIVYTGPIKVQQMKNFIDSLVASLSAAVSNLVKILKDTAAIDLETRQKFGVLDVTSRKWLIKPTAKSHAWGVVETHARKYHVALLEYDEQGVVTCDDWRRVAVSSESVVYSDMAKLRTLRRLLRNGEPHVSSAKVVLVDGVPGCGKTKEILSRVNFDEDLILVPGKQAAEMIRRRANSSGIIVATKDNVKTVDSFMMNFGKSTRCQFKRLFIDEGLMLHTGCVNFLVAMSLCEIAYVYGDTQQIPYINRVSGFPYPAHFAKLEVDEVETRRTTLRCPADVTHYLNRRYEGFVMSTSSVKKSVSQEMVGGAAVINPISKPLHGKILTFTQSDKEALLSRGYSDVHTVHEVQGETYSDVSLVRLTPTPVSIIAGDSPHVLVALSRHTCSLKYYTVVMDPLVSIIRDLEKLSSYLLDMYKVDAGTQXQLQIDSVFKGSNLFVAAPKTGDISDMQFYYDKCLPGNSTMMNNFDAVTMRLTDISLNVKDCILDMSKSVAAPKDQIKPLIPMVRTAAEMPRQTGLLENLVAMIKRNFNAPELSGIIDIENTASLVVDKFFDSYLLKEKRKPNKNVSLFSRESLNRWLEKQEQVTIGQLADFDFVDLPAVDQYRHMIKAQPKQKLDTSIQTEYPALQTIVYHSKKINAIFGPLFSELTRQLLDSVDSSRFLFFTRKTPAQIEDFFGDLDSHVPMDVLELDISKYDKSQNEFHCAVEYEIWRRLGFEDFLGEVWKQGHRKTTLKDYTAGIKTCIWYQRKSGDVTTFIGNTVIIAACLASMLPMEKIIKGAFCGDDSLLYFPKGCEFPDVQHSANLMWNFEAKLFKKQYGYFCGRYVIHHDRGCIVYYDPLKLISKLGAKHIKDWEHLEEFRRSLCDVAVSLNNCAYYTQLDDAVWEVHKTAPPGSFVYKSLVKYLSDKVLFRSLFIDGSSC

SEQ ID NO: 5 - DNA, 3351 bp, Tobacco mosaic virus
  CDS (1)..(3348)
  misc_feature (1096)..(1096): n is “t”, “c”, “a” or “g”,  except when nucleotide 1097 is “t” and nucleotide 1098 is “t” or “c”, n cannot be “t”
atggcatacacacagacagctaccacatcagctttgctggacactgtccgaggaaacaactccttggtcaatgatctagcaaagcgtcgtctttacgacacagcggttgaagagtttaacgctcgtgaccgcaggcccaaagtgaacttttcaaaagtaataagcgaggagcagacgcttattgctacccgggcgtatccagaattccaaattacattttataacacgcaaaatgccgtgcattcgcttgcaggtggattgcgatctttagaactggaatatctgatgatgcaaattccctacggatcattgacttatgacataggcgggaattttgcatcgcatctgttcaagggacgagcatatgtacactgctgcatgcccaacctggacgttcgagacatcatgcggcatgaaggccagaaagacagtattgaactatacctttctaggctagagagagggggaaaaacagtccccaacttccaaaaggaagcatttgacagatacgcagaaattcctgaagacgctgtctgtcacaatactttccagacatgcgaacatcagccgatgcaacaatcaggcagagtgtatgccattgcgctacacagcatatatgacatacccgctgatgagttcggggcagcactcttgaggaaaaatgtccatacgtgctatgccgctttccacttctctgagaacctgcttcttgaagattcatacgtcaatctggacgaaatcaacgcgtgtttttcgcgcgatggagacaagttgaccttttcttttgcatcagagagtactcttaattactgtcatagttattctaatattcttaagtatgtgtgcaaaacttacttcccggcctctaatagagaggtttacatgaaggagtttttagtcaccagggttaatacctggttttgtaagttttctagaatagatacttttcttttgtacaaaggtgtggcccataaaggtgtagatagtgagcagttttatactgcaatggaagacgcatggcattacaaaaagactcttgcaatgtgcaacagcgagagaatcctccttgaggattcatcaacagtcaattactggnnncccgaaatgagggatatggtcatcgtaccattattcgacatttctttggagactagtaagaggacgcgcaaggaagtcttagtgtccaaggatttcgtgtttacagtgcttaaccacattcgaacataccaggcaaaagctcttacatacgtaaatgttttgtccttcgtcgaatcgattcgatcgagggtaatcattaacggtgtgacagcgaggtccgaatgggatgtggacaaatctttgttacaatccttgtccatgacgttttacctgcatactaagcttgccgttctaaaggatgacttactgattagcaagtttagtctcggttcgaaaacggtgtgccagcatgtgtgggatgagatttcactggcgtttgggaacgcatttccctccgtgaaagagaggctcttgaacaggaaacttatcagagtggcaggcgacgcactagagatcagggtgcctgatctatatgtgaccttccacgaccgattagtgactgagtacaaggcctctgtggacatgcctgcgcttgacattaggaagaagatggaagaaacggaagtgatgtacaatgcactttcagagttatcggtgttaagggagtctgacaaattcgatgttgatgttttttcccagatgtgccaatctttggaagttgacgcaatgacggcagcgaaggttatagtcgcggtcatgagcaataagagcggtctgactctcacatttgaacgacctactgaggcgaatgttgcgctagctttacaggatcaagaaaaggcttcagaaggtgctttggtagttacctcaagagaagttgaagaaccgtccatgaagggttcgatggccagaggagagttacaattagctggtcttgctggagatcatccggagtcgtcctattctaggaacgaggagatagagtctttagagcagtttcatatggcaacggcagattcgttaattcgtaagcagatgagctcgattgtgtacacgggtccgattaaagttcagcaaatgaaaaactttatcgatagcctggtagcatcactatctgctgcggtgtcgaatctcgtcaagatcctcaaagatacagctgctattgaccttgaaacccgtcaaaagtttggagtcttggatgttacatctaggaagtggttaattaaaccaacggccaagagtcatgcatggggtgttgttgaaacccacgcgaggaagtatcatgtggcgcttctggaatatgatgagcagggtgtggtgacatgcgatgattggagaagagtagctgtcagctctgagtctgttgtttattccgacatggcgaaactcagaactctgcgcagactgcttcgaaacggagaaccgcatgtcagtagcgcaaaggttgttcttgtggacggagttccgggctgtggaaaaaccaaagaaattctttccagggttaattttgatgaagatctaattttagtacctgggaagcaagctgctgaaatgatcagaagacgtgcgaattcctcagggattattgtggccacgaaggacaacgttaaaaccgttgattctttcatgatgaattttgggaaaagcacacgctgtcagttcaagaggttattcattgatgaagggttgatgttgcatactggttgtgttaattttcttgtggcgatgtcattgtgcgaaattgcatatgtttacggagacacacagcagattccatacatcaatagagtttcaggattcccgtaccccgcccattttgccaaattggaagttgacgaggtggagacacgcagaactactctccgttgtccagccgatgtcacacattatctgaacaggagatatgagggctttgtcatgagcacttcttcggttaaaaagtctgtttcgcaggagatggtcggcggagccgccgtgatcaatccgatctcaaaacccttgcatggcaagatcctgacttttacccaatcggataaagaagctctgctttcaagagggtattcagatgttcacactgtgcatgaagtgcaaggcgagacatactctgatgtttcactagttaggctaacccctacaccagtctccatcattgcaggagacagcccgcatgttttggtcgcattgtcaaggcacacctgttcgctcaagtactacactgttgttatggatcctttagttagtatcattagagatctagagaaacttagctcgtacttgttagatatgtataaggtcgatgcaggaacacaatag

SEQ ID NO: 6 - protein, 1116 aa, Tobacco mosaic virus
  misc_feature (366)..(366): The ′Xaa′ at location 366 stands for any amino acid except Phe.
MAYTQTATTSALLDTVRGNNSLVNDLAKRRLYDTAVEEFNARDRRPKVNFSKVISEEQTLIATRAYPEFQITFYNTQNAVHSLAGGLRSLELEYLMMQIPYGSLTYDIGGNFASHLFKGRAYVHCCMPNLDVRDIMRHEGQKDSIELYLSRLERGGKTVPNFQKEAFDRYAEIPEDAVCHNTFQTCEHQPMQQSGRVYAIALHSIYDIPADEFGAALLRKNVHTCYAAFHFSENLLLEDSYVNLDEINACFSRDGDKLTFSFASESTLNYCHSYSNILKYVCKTYFPASNREVYMKEFLVTRVNTWFCKFSRIDTFLLYKGVAHKGVDSEQFYTAMEDAWHYKKTLAMCNSERILLEDSSTVNYWXPEMRDMVIVPLFDISLETSKRTRKEVLVSKDFVFTVLNHIRTYQAKALTYVNVLSFVESIRSRVIINGVTARSEWDVDKSLLQSLSMTFYLHTKLAVLKDDLLISKFSLGSKTVCQHVWDEISLAFGNAFPSVKERLLNRKLIRVAGDALEIRVPDLYVTFHDRLVTEYKASVDMPALDIRKKMEETEVMYNALSELSVLRESDKFDVDVFSQMCQSLEVDAMTAAKVIVAVMSNKSGLTLTFERPTEANVALALQDQEKASEGALVVTSREVEEPSMKGSMARGELQLAGLAGDHPESSYSRNEEIESLEQFHMATADSLIRKQMSSIVYTGPIKVQQMKNFIDSLVASLSAAVSNLVKILKDTAAIDLETRQKFGVLDVTSRKWLIKPTAKSHAWGVVETHARKYHVALLEYDEQGVVTCDDWRRVAVSSESVVYSDMAKLRTLRRLLRNGEPHVSSAKVVLVDGVPGCGKTKEILSRVNFDEDLILVPGKQAAEMIRRRANSSGIIVATKDNVKTVDSFMMNFGKSTRCQFKRLFIDEGLMLHTGCVNFLVAMSLCEIAYVYGDTQQIPYINRVSGFPYPAHFAKLEVDEVETRRTTLRCPADVTHYLNRRYEGFVMSTSSVKKSVSQEMVGGAAVINPISKPLHGKILTFTQSDKEALLSRGYSDVHTVHEVQGETYSDVSLVRLTPTPVSIIAGDSPHVLVALSRHTCSLKYYTVVMDPLVSIIRDLEKLSSYLLDMYKVDAGTQ

SEQ ID NO: 7 - DNA, 4834 bp, Tobacco mosaic virus
  misc_feature (1096)..(1096): n is “t”, “c”, “a” or “g”, except when nucleotide 1097 is “t” and nucleotide 1098 is “t” or “c”, n cannot be “t”
atggcatacacacagacagctaccacatcagctttgctggacactgtccgaggaaacaactccttggtcaatgatctagcaaagcgtcgtctttacgacacagcggttgaagagtttaacgctcgtgaccgcaggcccaaagtgaacttttcaaaagtaataagcgaggagcagacgcttattgctacccgggcgtatccagaattccaaattacattttataacacgcaaaatgccgtgcattcgcttgcaggtggattgcgatctttagaactggaatatctgatgatgcaaattccctacggatcattgacttatgacataggcgggaattttgcatcgcatctgttcaagggacgagcatatgtacactgctgcatgcccaacctggacgttcgagacatcatgcggcatgaaggccagaaagacagtattgaactatacctttctaggctagagagagggggaaaaacagtccccaacttccaaaaggaagcatttgacagatacgcagaaattcctgaagacgctgtctgtcacaatactttccagacatgcgaacatcagccgatgcaacaatcaggcagagtgtatgccattgcgctacacagcatatatgacatacccgctgatgagttcggggcagcactcttgaggaaaaatgtccatacgtgctatgccgctttccacttctctgagaacctgcttcttgaagattcatacgtcaatctggacgaaatcaacgcgtgtttttcgcgcgatggagacaagttgaccttttcttttgcatcagagagtactcttaattactgtcatagttattctaatattcttaagtatgtgtgcaaaacttacttcccggcctctaatagagaggtttacatgaaggagtttttagtcaccagggttaatacctggttttgtaagttttctagaatagatacttttcttttgtacaaaggtgtggcccataaaggtgtagatagtgagcagttttatactgcaatggaagacgcatggcattacaaaaagactcttgcaatgtgcaacagcgagagaatcctccttgaggattcatcaacagtcaattactggnnncccgaaatgagggatatggtcatcgtaccattattcgacatttctttggagactagtaagaggacgcgcaaggaagtcttagtgtccaaggatttcgtgtttacagtgcttaaccacattcgaacataccaggcaaaagctcttacatacgtaaatgttttgtccttcgtcgaatcgattcgatcgagggtaatcattaacggtgtgacagcgaggtccgaatgggatgtggacaaatctttgttacaatccttgtccatgacgttttacctgcatactaagcttgccgttctaaaggatgacttactgattagcaagtttagtctcggttcgaaaacggtgtgccagcatgtgtgggatgagatttcactggcgtttgggaacgcatttccctccgtgaaagagaggctcttgaacaggaaacttatcagagtggcaggcgacgcactagagatcagggtgcctgatctatatgtgaccttccacgaccgattagtgactgagtacaaggcctctgtggacatgcctgcgcttgacattaggaagaagatggaagaaacggaagtgatgtacaatgcactttcagagttatcggtgttaagggagtctgacaaattcgatgttgatgttttttcccagatgtgccaatctttggaagttgacgcaatgacggcagcgaaggttatagtcgcggtcatgagcaataagagcggtctgactctcacatttgaacgacctactgaggcgaatgttgcgctagctttacaggatcaagaaaaggcttcagaaggtgctttggtagttacctcaagagaagttgaagaaccgtccatgaagggttcgatggccagaggagagttacaattagctggtcttgctggagatcatccggagtcgtcctattctaggaacgaggagatagagtctttagagcagtttcatatggcaacggcagattcgttaattcgtaagcagatgagctcgattgtgtacacgggtccgattaaagttcagcaaatgaaaaactttatcgatagcctggtagcatcactatctgctgcggtgtcgaatctcgtcaagatcctcaaagatacagctgctattgaccttgaaacccgtcaaaagtttggagtcttggatgttacatctaggaagtggttaattaaaccaacggccaagagtcatgcatggggtgttgttgaaacccacgcgaggaagtatcatgtggcgcttctggaatatgatgagcagggtgtggtgacatgcgatgattggagaagagtagctgtcagctctgagtctgttgtttattccgacatggcgaaactcagaactctgcgcagactgcttcgaaacggagaaccgcatgtcagtagcgcaaaggttgttcttgtggacggagttccgggctgtggaaaaaccaaagaaattctttccagggttaattttgatgaagatctaattttagtacctgggaagcaagctgctgaaatgatcagaagacgtgcgaattcctcagggattattgtggccacgaaggacaacgttaaaaccgttgattctttcatgatgaattttgggaaaagcacacgctgtcagttcaagaggttattcattgatgaagggttgatgttgcatactggttgtgttaattttcttgtggcgatgtcattgtgcgaaattgcatatgtttacggagacacacagcagattccatacatcaatagagtttcaggattcccgtaccccgcccattttgccaaattggaagttgacgaggtggagacacgcagaactactctccgttgtccagccgatgtcacacattatctgaacaggagatatgagggctttgtcatgagcacttcttcggttaaaaagtctgtttcgcaggagatggtcggcggagccgccgtgatcaatccgatctcaaaacccttgcatggcaagatcctgacttttacccaatcggataaagaagctctgctttcaagagggtattcagatgttcacactgtgcatgaagtgcaaggcgagacatactctgatgtttcactagttaggctaacccctacaccagtctccatcattgcaggagacagcccgcatgttttggtcgcattgtcaaggcacacctgttcgctcaagtactacactgttgttatggatcctttagttagtatcattagagatctagagaaacttagctcgtacttgttagatatgtataaggtcgatgcaggaacacaatagcaattacagattgactcggtgttcaaaggttccaatctttttgtggcagcgccaaagactggtgatatttctgatatgcagttttactatgataagtgtctcccaggcaacagcaccatgatgaataattttgatgctgttaccatgaggttgactgacatttcattgaatgtcaaagattgcatattggatatgtctaagtctgttgctgcgcctaaggatcaaatcaaaccactaatacctatggtacgaacggcggcagaaatgccacgccagactggactattggaaaatttagtggcgatgattaaaaggaactttaacgcacccgagttgtctggcatcattgatattgaaaatactgcatctttagttgtagataagtttttcgatagttatttgcttaaagaaaaaagaaaaccaaataaaaatgtttctttgttcagtagagagtctctcaatagatggttagaaaagcaggaacaggtaacaataggccagctcgcagattttgattttgtagatttgccagcagttgatcagtacagacacatgatcaaagcacaacccaagcaaaaattggacacttcaatccaaacggagtacccggctttgcagacgattgtgtaccattcgaaaaagatcaatgcaatatttggcccgttgtttagtgagcttactaggcaattactggacagtgttgattcgagcagatttttgtttttcacaagaaagacaccagcgcagattgaggatttcttcggagatctcgacagtcatgtgccgatggatgtcttggagctggatatatcaaaatacgacaaatctcagaatgaattccactgtgcagtagaatacgagatttggcgaagattgggttttgaagacttcttgggagaagtttggaaacaagggcatagaaagaccaccctcaaggattataccgcaggtatcaaaacttgcatctggtatcaaagaaagagtggggacgtcacgacattcattggaaacactgtgatcattgctgcatgtttggcctcgatgcttccgatggagaaaataatcaaaggagccttttgtggtgacgatagtctgctgtacttcccaaagggttgtgagtttccggatgtgcaacactccgcgaatcttatgtggaattttgaagcaaaactgtttaaaaaacagtatggatacttttgcggaagatatgtaatacatcacgacagaggatgcattgtgtattacgatcccctaaagttgatctcgaaacttggcgctaaacacatcaaggattgggaacacttggaggagttcagaaggtctctttgtgatgttgctgtttcgttgaacaattgtgcgtattatacacagttggacgacgctgtatgggaggttcataagaccgcccctccaggttcgtttgtttataaaagtctggtgaagtatttgtctgataaagttctttttagaagtttgtttatag

SEQ ID NO: 8 - protein, 1616 aa, Tobacco mosaic virus
  MISC_FEATURE (366)..(366): The ′Xaa′ at location 366 stands for any amino acid except Phe.
MAYTQTATTSALLDTVRGNNSLVNDLAKRRLYDTAVEEFNARDRRPKVNFSKVISEEQTLIATRAYPEFQITFYNTQNAVHSLAGGLRSLELEYLMMQIPYGSLTYDIGGNFASHLFKGRAYVHCCMPNLDVRDIMRHEGQKDSIELYLSRLERGGKTVPNFQKEAFDRYAEIPEDAVCHNTFQTCEHQPMQQSGRVYAIALHSIYDIPADEFGAALLRKNVHTCYAAFHFSENLLLEDSYVNLDEINACFSRDGDKLTFSFASESTLNYCHSYSNILKYVCKTYFPASNREVYMKEFLVTRVNTWFCKFSRIDTFLLYKGVAHKGVDSEQFYTAMEDAWHYKKTLAMCNSERILLEDSSTVNYWXPEMRDMVIVPLFDISLETSKRTRKEVLVSKDFVFTVLNHIRTYQAKALTYVNVLSFVESIRSRVIINGVTARSEWDVDKSLLQSLSMTFYLHTKLAVLKDDLLISKFSLGSKTVCQHVWDEISLAFGNAFPSVKERLLNRKLIRVAGDALEIRVPDLYVTFHDRLVTEYKASVDMPALDIRKKMEETEVMYNALSELSVLRESDKFDVDVFSQMCQSLEVDAMTAAKVIVAVMSNKSGLTLTFERPTEANVALALQDQEKASEGALVVTSREVEEPSMKGSMARGELQLAGLAGDHPESSYSRNEEIESLEQFHMATADSLIRKQMSSIVYTGPIKVQQMKNFIDSLVASLSAAVSNLVKILKDTAAIDLETRQKFGVLDVTSRKWLIKPTAKSHAWGVVETHARKYHVALLEYDEQGVVTCDDWRRVAVSSESVVYSDMAKLRTLRRLLRNGEPHVSSAKVVLVDGVPGCGKTKEILSRVNFDEDLILVPGKQAAEMIRRRANSSGIIVATKDNVKTVDSFMMNFGKSTRCQFKRLFIDEGLMLHTGCVNFLVAMSLCEIAYVYGDTQQIPYINRVSGFPYPAHFAKLEVDEVETRRTTLRCPADVTHYLNRRYEGFVMSTSSVKKSVSQEMVGGAAVINPISKPLHGKILTFTQSDKEALLSRGYSDVHTVHEVQGETYSDVSLVRLTPTPVSIIAGDSPHVLVALSRHTCSLKYYTVVMDPLVSIIRDLEKLSSYLLDMYKVDAGTQXQLQIDSVFKGSNLFVAAPKTGDISDMQFYYDKCLPGNSTMMNNFDAVTMRLTDISLNVKDCILDMSKSVAAPKDQIKPLIPMVRTAAEMPRQTGLLENLVAMIKRNFNAPELSGIIDIENTASLVVDKFFDSYLLKEKRKPNKNVSLFSRESLNRWLEKQEQVTIGQLADFDFVDLPAVDQYRHMIKAQPKQKLDTSIQTEYPALQTIVYHSKKINAIFGPLFSELTRQLLDSVDSSRFLFFTRKTPAQIEDFFGDLDSHVPMDVLELDISKYDKSQNEFHCAVEYEIWRRLGFEDFLGEVWKQGHRKTTLKDYTAGIKTCIWYQRKSGDVTTFIGNTVIIAACLASMLPMEKIIKGAFCGDDSLLYFPKGCEFPDVQHSANLMWNFEAKLFKKQYGYFCGRYVIHHDRGCIVYYDPLKLISKLGAKHIKDWEHLEEFRRSLCDVAVSLNNCAYYTQLDDAVWEVHKTAPPGSFVYKSLVKYLSDKVLFRSLFIDGSSC

SEQ ID NO: 9 - protein, 9 aa, Alfalfa mosaic virus
  PEPTIDE (1)..(9)
SCAWYNRVK

SEQ ID NO: 10 - protein, 9 aa, Brome mosaic virus
  PEPTIDE (1)..(9)
HCVWFEDIS

SEQ ID NO: 11 - protein, 9 aa, Citrus leaf rugose virus
  PEPTIDE (1)..(9)
SCAWLSSLR

SEQ ID NO: 12 - protein, 9 aa, cucumber mosaic virus
  PEPTIDE (1)..(9)
HCIWFPSMK

SEQ ID NO: 13 - protein, 9 aa, Sunn-hemp mosaic virus
  PEPTIDE (1)..(9)
FNVYFPNAK

SEQ ID NO: 14 - protein, 10 aa, Tobacco mosaic virus
  PEPTIDE (1)..(10)
SVNYWFPKMR

SEQ ID NO: 15 - protein, 9 aa, Tobacco rattle virus
  PEPTIDE (1)..(9)
VEKQFMDKC

SEQ ID NO: 16 - protein, 9 aa, Turnip vein-clearing virus
  PEPTIDE (1)..(9)
LNFWFPKVR

SEQ ID NO: 17 - protein, 16 aa, Tobacco mosaic virus
  PEPTIDE (1)..(16)
SSVNYWFPKMRAPEKA

SEQ ID NO: 18 - protein, 16 aa, Tobacco mosaic virus
  PEPTIDE (1)..(16)
GTVNYWFPEMRVAKRT

SEQ ID NO: 19 - protein, 16 aa, Tobacco mosaic virus
  PEPTIDE (1)..(16)
GSVNYWFPEMRVAKRT

SEQ ID NO: 20 - protein, 16 aa, Tobacco mosaic virus
  PEPTIDE (1)..(16)
GSVNYWAPEMRVAKRT

SEQ ID NO: 21 - protein, 16 aa, Tobacco mosaic virus
  PEPTIDE (1)..(16)
GSVNYWYPEMRVAKRT

SEQ ID NO: 22 - DNA, 37 bp, Tobacco mosaic virus
  misc_feature (1)..(37): PCR primer
ctcatttcgggagcccagtaattgactgatgatgaat

SEQ ID NO: 23 - DNA, 33 bp, Tobacco mosaic virus
  misc_feature (1)..(33): PCR primer
tttcgggataccagtaattgactgatgatgaat

SEQ ID NO: 24 - DNA, 37 bp, Tobacco mosaic virus
  misc_feature (1)..(37): PCR primer
ccatgccatggcgctcgagatggcatacacacagaca

SEQ ID NO: 25 - DNA, 30 bp, Tobacco mosaic virus
  misc_feature (1)..(30): PCR primer
cccttgctcaccatttgtgttcctgcatcg

SEQ ID NO: 26 - DNA, 30 bp, Plasmid pEGFP
  misc_feature (1)..(30): PCR primer
atgcaggaacacaaatggtgagcaagggcg

SEQ ID NO: 27 - DNA, 34 bp, Plasmid pEGFP
  misc_feature (1)..(34): PCR primer
ccatgccatggctcgagttacttgtacagctcgt

(19) United States
(12) Patent Application Publication
(10) Pub. No.: US 2004/0191911 A1
(43) Pub. Date: Sep. 30, 2004

(54) Method for altering degradation of engineered protein in plant cells

(76) Inventors: Richard S Nelson, Oklahoma, OK (US); Yiming Bao, Germantown, MD (US); Ning-Hui Cheng, Houston, TX (US)

Correspondence Address: FULBRIGHT & JAWORSKI, 600 CONGRESS AVENUE SUITE 1900, AUSTIN, TX, 78701, US

(21) Appl. No.: 10/332,284

(22) PCT Filed: Jul. 16, 2001
(86) PCT No.: PCT/US01/22390

Related U.S. Application Data
(60) Provisional application No. 60/218,504, filed on Jul. 15, 2000.

(51) Int. Cl. C12N 15/82; C12N 5/04

(52) U.S. Cl. 435/468; 435/69.1; 435/419

(57) ABSTRACT

A method of altering degradation of heterologous proteins in transgenic plants has now been found that utilizes ER-localizing proteins of plant viruses as part of a fusion protein. An engineered fusion protein is protected from degradation by a viral ER-localizing protein, and made more susceptible to degradation by certain mutant viral proteins that fail to localize to the ER.

### CROSS-REFERENCE TO RELATED APPLICATION

This application claims the benefit of U. S. Provisional Patent Application Serial No. 60/218,504, filed Jul. 15, 2000.

### TECHNICAL FIELD OF INVENTION

This invention relates to a method of altering the rate of degradation of proteins in plant cells.

### BACKGROUND OF THE INVENTION

Intracellular protein concentration is influenced by many factors, including the rates of transcription, translation, and degradation. When cells are engineered to express a protein from a transgene, robust and stable expression may be desired. In other circumstances, limited accumulation of a protein from a transgene may be desired. Being able to modulate an engineered protein's rate of degradation has numerous applications.

One advantage of being able to manipulate a protein's degradation rate is to increase its intracellular concentration to study its function. After translation, protein levels are controlled by protease activity (Vierstra, 1996) which can limit the accumulation of proteins under study to levels that prevent their biochemical characterization. Another advantage to increasing a selected protein's intracellular concentration is that it may enhance the accumulation of foreign proteins with beneficial traits in transgenic plants (Vierstra, 1996). As a contrast, there may also be advantages to enhancing a protein's degradation. The identification of sequences that lead to faster degradation of proteins will benefit researchers interested in repressing accumulation of unwanted endogenous proteins that interfere with important agronomic processes (Vierstra, 1996). Interest in methods to regulate protein accumulation is reflected by the approaches that have been previously reported. One method includes modifying the primary sequence to remove domains conferring instability, and another method is to inhibit proteases (reviewed in Vierstra, 1996.) In another instance, ubiquitin, a stable protein, was fused to a poorly expressed protein to enhance the expression of the latter (Eker et al. 1989).

Non-host proteins are produced in viral-infected plants. During tobacco mosaic virus (TMV) infection, two such proteins are the 126 kDa protein and the 183 kDa protein, a read-through product containing the 126 kDa protein sequence. Description of these proteins in the prior art indicate that they play a role in replication. Approximately 10% of the 126 kDa protein heterodimerizes with essentially all of the 183 kDa protein in the plant cell, even though the 183 kDa protein alone is capable of replicating the virus in infected cells (Watanabe et al., 1999; Lewandowski and Dawson, 2000). Both proteins are reportedly required for efficient TMV replication in vivo (Osman and Buck, 1996; Watanabe et al., 1999). In fact, the 126 kDa/183 kDa proteins were found with other TMV and host plant factors in the viral replication complex (Heinlein et al., 1998). Additionally, the 126 kDa/183 kDa proteins have putative methyltransferase and helicase domains. Furthermore, the 183 kDa protein contains a carboxy terminal domain required for RNA-dependent RNA polymerase activity.

Although the role of the 126 kDa protein and/or the 183 kDa protein of TMV is thought in the prior art to be replication, its intracellular localization was unknown. Mas and Beachy (1999) observed that the 126 kDa protein of TMV co-localizes with viral RNA in subcellular bodies and with luminal binding protein (BiP), an endoplasmic reticulum (ER)-specific protein, in infected plants. Although these observations suggested to Mas and Beachy that the 126 kDa protein and/or the 183 kDa protein of TMV localizes to the ER, the localization signal of the proteins was not identified.

Comparing the 126 kDa protein and/or the 183 kDa protein of TMV to another species suggested in the prior art that the proteins may localize to the ER. Brome mosaic virus BMV), a virus related to Tobacco mosaic virus (TMV), possesses a protein believed to be homologous in function to the 126 kDa protein of TMV, although its overall sequence identity with the TMV protein is 13%. Previous publications determined that the BMV 1a protein localized to the ER during infection of barley cells and that, in the absence of other viral proteins, it localized to the ER in yeast (Restrepo-Hartwig and Ahlquist 1999). Therefore, the BMV 1a protein, like its putative TMV homolog, may localize to specific subcellular locations. In additional to localizing to the endoplasmic reticulum in yeast, the 1a protein also stabilized viral RNA (Sullivan and Ahlquist, 1999) and decreased the viral RNA translation (Janda and Ahlquist, 1998).

The post-translational regulation of the 126 kDa protein and/or the 183 kDa protein of TMV has also been studied in the prior art, but only with ambiguous results. Previous reports indicated that 26S proteasome inhibitors had no significant effect on 126 kDa or 183 kDa protein accumulation in plant cell suspensions infected with TMV (Reichel and Beachy, 2000). In late stages of TMV infection, what little effect occurred indicated that the protein was more susceptible to degradation in the presence of the 26S proteasome inhibitor. From these results it appeared that induction of 26S proteasome activity had no significant influence on the degradation of the 126 kDa protein. Importantly, the ability of the 126 kDa protein to stabilize its expression in the absence of other viral proteins was not tested in these studies by Reichel and Beachy.

There is a desire and a need in agronomic biotechnology to modulate the expression level of engineered proteins. Expression in cells engineered to express a protein from a transgene may be robust and stable; in other circumstances, limited accumulation of a protein from a transgene may be desired. To fulfill that need, we have developed a ubiquitin-fusion—independent system in which the degradation—and hence the protein level—of an engineered protein can be modulated in plant cells.

### BRIEF DESCRIPTION OF THE DRAWINGS

FIG. 1 is a partial protein sequence alignment (amino acids 361-370 of SEQ ID NO:2 and SEQ ID NO:4) of the TMV 126/183 kDa protein and its functional analogs from Sindbis—like plant viruses. The conserved “WFP” motif is boxed and the amino acids in bold type are identical to amino acids 365-367 of SEQ ID NO:2 and SEQ ID NO:4. The underlined letters (serine 361 and lysine 368) show the amino acids in the TMV U1 strain that are different from that of the M^{IC} strain. AMV: alfalfa mosaic virus (SEQ ID NO:9); BMV: brome mosaic virus (SEQ ID NO:10); CiLRV: citrus leaf rugose virus (SEQ ID NO:11); CMV: cucumber mosaic virus (SEQ ID NO:12); SHMV: sunn-hemp mosaic virus (SEQ ID NO:13); TMV: tobacco mosaic virus U1 strain (SEQ ID NO:14); TRV: tobacco rattle virus (SEQ ID NO:15); and TVCV: turnip vein clearing virus (SEQ ID NO:16).

FIG. 2A depicts the genome organization of TMV. Three open reading frames (ORFs) which encode the 126 kDa protein (1-3348 of SEQ ID NO:1) and the read-through 183 kDa protein (1-4831 of SEQ ID NO:3), the movement protein (horizontal stripes), and the coat protein (dotted). A black arrowhead indicates the location of the leaky amber stop codon (UAG) within the replicase ORF. The methyltransferase domain of the 126/183 kDa protein is represented with vertical stripes, beginning at nucleotide 142 of SEQ ID NO:1 and ending at nucleotide 900 of SEQ ID NO:1. The helicase domain of the 126/183 kDa protein is represented with diamonds, beginning at nucleotide 2362 of SEQ ID NO:1 and ending at nucleotide 3249 of SEQ ID NO:1. GDD (white) is a motif present in viral RNA-dependent RNA polymerase. Domains I and II of the 126/183 kDa protein each have 4 amino acid mutations that were identified to control the phenotype difference between the TMV U1 strain, which causes severe symptoms, and the cloned Masked strain of TMV (M^{IC}), which causes mild symptoms. Nucleotide numbers in the figure refer to the entire genome of TMV, Genbank Accession No. AF273221.

FIG. 2B depicts the different amino acids present within Domains I and II of the 126/183 kDa protein and the resulting symptoms of the viruses. The following sequences were aligned: TMV-U1 (SEQ ID NO:17), the parental TMV-M^{IC} (SEQ ID NO:18), and the site-directed mutant viruses studied, TMV-M^{IC}2 (SEQ ID NO:19), TMV-WAP (SEQ ID NO:20), and TMV-WYP (SEQ ID NO:21). Among all sequences, the amino acids in 8 positions (four mutations in each of two domains) were determined to vary: 325, 360, 367, 416, 587, 601, 668, and 747, referring to the entire genome of TMV, Genbank Accession No. AF273221.

FIG. 3A depicts the lesion response (pictorially white spots) on a N. tabacum Xanthi “NN” leaf challenged with WFP and WYP viruses. Each half of the leaf was inoculated with either the WFP or WYP virus and the plant grown at 24° C. for ten days. The side of the leaf inoculated with the WFP virus resulted in a slightly larger lesions than the side of the leaf infected with the WYP virus.

FIG. 3B depicts the lesion response (pictorially white spots) on a N. tabacum Xanthi “NN” leaf challenged with WFP and WYP viruses. Each half of the leaf was inoculated with either the WFP or WYP virus and the plant grown at 32° C. for three days. The temperature was then decreased to 24° C. for another seven days. The size of lesions on the WFP virus—treated side of the leaf increased relative to the side of the leaf treated with the WYP virus.

FIGS. 4A-H depict immunolabeling experiments in N. tabacum BY-2 protoplasts. All images for FIGS. 4A-H were captured by confocal laser scanning microscopy using a previously described procedure (Cheng, et al., 2000). Bar=20 μM. FIG. 4A depicts immunolabeling of the 126 kDa protein in an N. tabacum BY-2 protoplast infected with the WFP virus. The pictorially light region indicates the presence and location of the 126 kDa protein. FIG. 4B depicts immunolabeling of BiP in an N. tabacum BY-2 protoplast infected with the WFP virus. The pictorially light region indicates the presence and location of the BiP protein. FIG. 4C depicts immunolabeling of the 126 kDa protein in an N. tabacum BY-2 protoplast infected with the WYP virus. The pictorially light region indicates the presence and location of the 126 kDa protein. FIG. 4D depicts immunolabeling of BiP in an N. tabacum BY-2 protoplast infected with the WYP virus. The pictorially light region indicates the presence and location of the BiP protein.

FIG. 4E depicts immunolabeling of the 126 kDa protein in an N. tabacum BY-2 protoplast infected with the M^{IC} virus. The pictorially light region indicates the presence and location of the 126 kDa protein. FIG. 4F depicts immunolabeling of BiP in an N. tabacum BY-2 protoplast infected with the M^{IC} virus. The pictorially light region indicates the presence and location of the BiP protein. FIG. 4G depicts immunolabeling of the 126 kDa protein in a mock-inoculated N. tabacum BY-2 protoplast. As expected, there is no detection of the 126 kDa protein. FIG. 4H depicts immunolabeling of BiP in a mock-inoculated N. tabacum BY-2 protoplast. The pictorially light region indicates the presence and location of the BiP protein that was not localized, unlike when 126 kDa protein from the WFP or M^{IC} virus was present.

FIG. 5A is a diagram of a portion of genetic constructs bombarded into host leaves for transient expression. Open arrows depict the enhanced 35 S promoter; the dotted box represents nucleotides 1-3348 of SEQ ID NO:1 (for construct 126F:GFP) that encodes the 126 kDa protein from TMV; the box with diagonal stripes represents the DNA encoding for GFP (EGFP, Clontech Laboratories, Palo, Alto, Calif.); and the filled arrow represents the mRNA termination sequence. The bolded letter in the sequence depicted in the 126 kDa protein indicates the amino acid differences among the constructs. For construct 126Y:GFP, nucleotides 1-3348 of SEQ ID NO:5 where nucleotides that encode amino acid 366 are “ata” were inserted, and for construct 126A:GFP, nucleotides 1-3348 of SEQ ID NO:5 where nucleotides that encode amino acid 366 are “agc” were inserted. Each genetic element with the exception of the nucleotides encoding the 126 kDa protein from TMV originated in the expression vector pRTL2 (Topfer, et al. 1987 and Restrepo-Hartwig, et al. 1990).

FIGS. 5B-5G depict transient expression of the WFP-containing 126 kDa:GFP fusion proteins in N. tabacum (N.t) and N. benthamiana (N.b) leaves. White color on black background indicates the presence of fused protein. At 16 hours post-bombardment, the WFP-containing 126 kDa:GFP fusion construct bombarded onto N. tabacum leaves has similar expression to that of the same construct inoculated on N. benthamiana leaves (FIGS. 5B and 5C). This trend continues through the 44 hour and 8 day time points (FIGS. 5D and 5E and FIGS. 5F and 5G, respectively).

FIGS. 5H-5M depict transient expression of the WAP-containing 126 kDa:GFP fusion proteins in N. tabacum (N.t) and N. benthamiana (N.b) leaves. White color on black background indicates the presence of fused protein. The WAP-containing 126 kDa:GFP fusion construct bombarded onto N. benthamiana leaves has similar expression 16 hours post-bombardment than the same construct inoculated on N. tabacum leaves (FIGS. 5H and 5I). At 44 hours post-bombardment, there is more 126 kDa:GFP fusion expression on N. benthamiana leaves than at 16 hours, but far less 126 kDa:GFP fusion expression on N. tabacum leaves than the previous time point (FIGS. 5J and 5K). By 8 days there is low expression of the 126 kDa:GFP fusion expression on N. benthamiana leaves and no expression on N. tabacum leaves (FIGS. 5L and 5M).

FIGS. 5N-5S depict transient expression of the WYP containing 126 kDa:GFP fusion proteins in N. tabacum (N.t) and N. benthamiana (N.b) leaves. White color on black background indicates the presence of fused protein. At 16 hours post-bombardment, there is similar expression of the WYP-containing 126 kDa:GFP fusion constructs in N. tabacum and N. benthamiana leaves (FIGS. 5N and 5O). At 44 hours post-bombardment, the expression of the WYP-containing 126 kDa:GFP fusion constructs on N. tabacum and N. benthamiana leaves appears similar and low (FIGS. 5P and 5Q). At 8 days post-bombardment, significant expression of the WYP-containing 126 kDa:GFP fusion constructs on N. benthamiana leaves remain, whereas there is little if any expression of the WYP-containing 126 kDa:GFP fusion constructs on N. tabacum leaves (FIGS. 5R and 5S).

FIGS. 6A-6H depict the resulting expression of the 126F:GFP (WFP-containing construct), 126Y:GFP (WYP-containing construct), and 126A:GFP (WAP-containing construct) in N. benthamiana protoplasts. Although fluorescent bodies are detected in all protoplasts electroporated with the fusion constructs (FIGS. 6A-6F), the size of the bodies is smaller in the protoplast electroporated with the 126A:GFP construct (FIG. 6A) than in the other protoplasts (FIGS. 6C and 6E) 7 hours after electroporation. At 24 hours after electroporation, the protoplasts expressing the 126F:GFP and 126Y:GFP constructs appear to have fewer, but larger fluorescent bodies (FIGS. 6D and 6F). The protoplasts expressing free GFP form no punctate bodies even after 24 hours (FIGS. 6G and 6H). Bar=10 μM.

FIG. 7A provides the quantities of large (>2 μM) fluorescent bodies per protoplast formed by the transiently expressed WFP-, WYP-, or WAP-containing fusion proteins in N. benthamiana protoplasts over time (means±SD). The bars with horizontal stripes represent the expression of the WFP-containing fusion construct. The bars with the vertical stripes represent the expression of the WYP-containing fusion construct. The bars with diagonal stripes represent the expression of the WAP-containing fusion construct. The number of large bodies in N. benthamiana protoplasts transiently expressing WFP-, WYP-, or WAP-containing fusion proteins does not significantly differ among treatments at 16-36 hours. However, after 48 hours N. benthamiana protoplasts transiently expressing the WFP-containing fusion protein have more large bodies than protoplasts transiently expressing the other fusion proteins and the difference exists at the 72 and 96 hour time points as well.

FIG. 7B provides the quantities of small (<2 μM) fluorescent bodies formed by the transiently expressed WFP-, WYP-, or WAP-containing fusion proteins in N. benthamiana protoplasts over time (means±SD). Generally, within each treatment the amounts of small fluorescent bodies decrease with time. Although there is no significant difference between treatments at each time point, the N. benthamiana protoplasts expressing the WYP-containing fusion protein appear to have a greater number of small bodies than the other treatments until the 96 hour time point.

FIG. 7C provides the ratio of small (<2 μM) fluorescent bodies to large (>2 μM) fluorescent bodies formed by the transiently expressed WFP-, WYP-, or WAP-containing fusion proteins in N. benthamiana protoplasts over time (means±SD). There does not appear to be significant differences between treatments at every time point, but the smallest ratio of small to large fluorescent bodies in N. benthamiana protoplasts have WFP-containing fusion proteins at 48-96 hours post-electroporation.

FIGS. 8A-8F depict the transient expression of WFP-containing fusion proteins in N. tabacum BY-2 protoplasts in the presence (FIGS. 8B, 8D, and 8F) or absence (FIGS. 8A, 8C, and 8E) of a ubiquitin pathway inhibitor, ALLN, over time (12, 24, and 48 hours). There is greater WFP-containing fusion protein expression in protoplasts treated with ALLN than without ALLN at every time point (compare FIG. 8A to FIG. 8B, FIG. 8C to FIG. 8D, and FIG. 8E to FIG. 8F). Bar=10 μM.

FIGS. 8G-8L depict the transient expression of WYP-containing fusion proteins in N. tabacum BY-2 protoplasts in the presence (FIGS. 8H, 8J, and 8L) or absence (FIGS. 8G, 8I, and 8K) of a ubiquitin pathway inhibitor, ALLN, over time (12, 24, and 48 hours). There is greater WFP-containing fusion protein expression in protoplasts treated with ALLN than without ALLN at every time point (compare FIG. 8G to FIG. 8H, FIG. 8I to FIG. 8J, and FIG. 8K to FIG. 8L). However, transient expression in the absence of ALLN peaked 24 hours post-electroporation with little expression at 48 hours post-electroporation. In BY-2 protoplasts expressing the WYP-containing fusion protein and treated with ALLN, the number of small bodies decreased as the large bodies increased in size over time (FIG. 8H, 8J and 8K). Bar=10 μM.

FIGS. 8M-8R depict the transient expression of WAP-containing fusion proteins in N. tabacum BY-2 protoplasts in the presence (FIGS. 8N, 8P, and 8R) or absence (FIGS. 8M, 8O, and 8Q) of a ubiquitin pathway specific inhibitor, ALLN, over time (12, 24, and 48 hours). At 12 and 24 hours post-electroporation, BY-2 protoplasts in the presence of ALLN transiently express more WAP-containing fusion protein than the time-matched, -ALLN protoplasts (compare FIG. 8M to FIG. 8N and FIG. 8O to FIG. 8P). Also, there was greater WAP-containing fusion protein expression in ALLN treated BY-2 protoplasts at 24 hours than at 12 hours post-electroporation (FIG. 8N and FIG. 8P). However, at 48 hours, there was no detectable WAP-containing fusion protein expression in BY-2 protoplasts in the absence of ALLN (FIG. 8Q) and only very little, but aggregated, expression in the presence of ALLN (FIG. 8R). Bar=10 μM.

### SUMMARY OF THE INVENTION

In one aspect, the invention is a method for decreasing the degradation rate of an engineered protein of interest in a plant cell comprising the steps a) constructing a vector comprising a nucleic acid fragment from position 1 to position 3348 of SEQ ID NO:1 fused to a nucleotide sequence encoding a protein of interest, the vector expressible in said plant cell; and b) introducing and expressing the vector in the plant cell to form a fused protein, wherein the degradation rate of the fused protein is less than the degradation rate of the engineered protein of interest in the plant cell or a plant cell of the same species. The vector may be integrated into the genome of said plant cell. The invention is furthermore a plant cell transformed according to the above method and a plant generated from the transformed plant cell.

In another aspect, the invention is a method for decreasing the degradation rate of an engineered protein of interest in a plant cell comprising the steps a) constructing a vector comprising a nucleic acid fragment from position 1 to position 4831 of SEQ ID NO:3 fused to a nucleotide sequence encoding a protein of interest, the vector expressible in said plant cell; and b) introducing and expressing the vector in the plant cell to form a fused protein, wherein the degradation rate of the fused protein is less than the degradation rate of the engineered protein of interest in the plant cell or a plant cell of the same species. The vector may be integrated into the genome of said plant cell. The invention is furthermore a plant cell transformed according to the above method and a plant generated from the transformed plant cell.

In another aspect, the invention is also a method for increasing the degradation rate of an engineered protein of interest in a plant cell comprising the steps a) constructing a vector comprising a nucleic acid fragment from position 1 to position 3348 of SEQ ID NO:5 fused to a nucleotide sequence encoding a protein of interest, the vector expressible in a plant cell; and b) introducing and expressing the vector in the plant cell to form a fused protein, wherein the degradation rate of the fused protein is less than the degradation rate of the engineered protein of interest in the plant cell or a plant cell of the same species. Nucleotides at positions 1096-1098 of SEQ ID NO:5 encode alanine or tyrosine. The vector may be integrated into the genome of said plant cell. The invention is furthermore a plant cell transformed according to the above method and a plant generated from the transformed plant cell.

In another aspect, the invention is also a method for increasing the degradation rate of an engineered protein of interest in a plant cell comprising the steps a) constructing a vector comprising a nucleic acid fragment from position 1 to position 4831 of SEQ ID NO:7 fused to a nucleotide sequence encoding a protein of interest, the vector expressible in a plant cell; and b) introducing and expressing the vector in the plant cell to form a fused protein, wherein the degradation rate of the fused protein is less than the degradation rate of the engineered protein of interest in the plant cell or a plant cell of the same species. Nucleotides at positions 1096-1098 of SEQ ID NO:7 encode alanine or tyrosine. The vector may be integrated into the genome of said plant cell. The invention is furthermore a plant cell transformed according to the above method and a plant generated from the transformed plant cell.

In another aspect, the invention is a purified nucleic acid comprising a nucleic acid fragment from position 1 to position 3348 of SEQ ID NO:1 fused to a DNA sequence encoding a protein of interest, wherein expression of said purified nucleic acid in a plant cell results in a fusion protein having increased stability when compared to the stability of said protein of interest engineered without fusion expressed in a plant cell of the same species. The invention is also the resulting fusion protein comprising SEQ ID NO:2 encoded by the purified nucleic acid comprising a nucleic acid fragment from position 1 to position 3348 of SEQ ID NO:1 fused to a DNA sequence encoding a protein of interest. Another embodiment of the invention is the vector comprised of SEQ ID NO:1 encoding SEQ ID NO:2, the plant cell transformed with the vector, and the plant generated with the transformed plant cell.

In another aspect, the invention is a purified nucleic acid comprising a nucleic acid fragment from position 1 to position 4831 of SEQ ID NO:3 fused to a DNA sequence encoding a protein of interest, wherein expression of said purified nucleic acid in a plant cell results in a fusion protein having increased stability when compared to the stability of said protein of interest engineered without fusion expressed in a plant cell of the same species. The invention is also the resulting fusion protein comprising SEQ ID NO:4 encoded by the purified nucleic acid comprising a nucleic acid fragment from position 1 to position 4831 of SEQ ID NO:3 fused to a DNA sequence encoding a protein of interest. Another embodiment of the invention is the vector comprised of SEQ ID NO:3 encoding SEQ ID NO:4, the plant cell transformed with the vector, and the plant generated with the transformed plant cell.

In another aspect, the invention is a purified nucleic acid comprising a nucleic acid fragment from position 1 to position 3348 of SEQ ID NO:5 fused to a DNA sequence encoding a protein of interest, wherein expression of said purified nucleic acid in a plant cell results in a fusion protein having increased or decreased stability when compared to the stability of said protein of interest engineered without fusion expressed in a plant cell of the same species. The purified nucleic acid comprising a nucleic acid fragment from position 1 to position 3348 of SEQ ID NO:5 fused to a DNA sequence encoding a protein of interest could also have increased or decreased stability when compared to the stability of the protein of interest fused to a nucleic acid fragment from position 1 to position 3348 of SEQ ID NO:1 expressed in a plant cell of the same species. Nucleotides at positions 1096-1098 of SEQ ID NO:5 encode alanine or tyrosine. The invention is also the resulting fusion protein comprising SEQ ID NO:6 encoded by the purified nucleic acid comprising a nucleic acid fragment from position 1 to position 3348 of SEQ ID NO:5 fused to a DNA sequence encoding a protein of interest. Another embodiment of the invention is the vector comprised of SEQ ID NO:5 encoding SEQ ID NO:6, the plant cell transformed with the vector, and the plant generated with the transformed plant cell.

In another aspect, the invention is also a purified nucleic acid comprising a nucleic acid fragment from position 1 to position 4831 of SEQ ID NO:7 fused to a DNA sequence encoding a protein of interest, wherein expression of said purified nucleic acid in a plant cell results in a fusion protein having increased or decreased stability when compared to the stability of said protein of interest engineered without fusion expressed in a plant cell of the same species. The purified nucleic acid comprising a nucleic acid fragment from position 1 to position 4831 of SEQ ID NO:7 fused to a DNA sequence encoding a protein of interest could also have increased or decreased stability when compared to the stability of the protein of interest fused to a nucleic acid fragment from position 1 to position 3348 of SEQ ID NO:1 expressed in a plant cell of the same species. Nucleotides at positions 1096-1098 of SEQ ID NO:5 encode alanine or tyrosine. The invention is also the resulting fusion protein comprising SEQ ID NO:8 encoded by the purified nucleic acid comprising a nucleic acid fragment from position 1 to position 4831 of SEQ ID NO:7 fused to a DNA sequence encoding a protein of interest. Another embodiment of the invention is the vector comprised of SEQ ID NO:7 encoding SEQ ID NO:8, the plant cell transformed with the vector, and the plant generated with the transformed plant cell.

In yet another aspect, the invention is a method for decreasing the degradation rate of an engineered protein of interest in a plant cell comprising the steps a) constructing a vector comprising a nucleic acid sequence that encodes a membrane binding protein from the Sindbis-like plant virus family fused to a nucleotide sequence encoding the protein of interest, the vector expressible in a plant cell; and b) introducing and expressing he vector in the plant cell to form a fused protein, wherein the degradation rate of the fused protein is less than the degradation rate of the engineered protein of interest in the plant cell or a plant cell of the same species. The Sindbis-like plant virus family contains “WFP” motif as depicted at amino acid position 365-367 of SEQ ID NO:2. The vector may be integrated into the genome of said plant cell. The Sindbis-like plant virus is alfalfa mosaic virus, brome mosaic virus, citrus leaf rugose virus, cucumber mosaic virus, sunn-hemp mosaic virus, tobacco mosaic virus, tobacco rattle virus, or turnip vein clearing virus. The invention further embodies a plant cell transformed according to this method and the plant generated from the transformed plant cell.

In another aspect, the invention also embodies a method for increasing the degradation rate of an engineered protein of interest in a plant cell comprising the steps a) constructing a vector comprising a nucleic acid sequence that encodes a membrane binding protein from the Sindbis-like plant virus family fused to a nucleotide sequence encoding the protein of interest, the vector expressible in a plant cell; and b) introducing and expressing he vector in the plant cell to form a fused protein, wherein the degradation rate of the fused protein is less than the degradation rate of the engineered protein of interest in the plant cell or a plant cell of the same species. The Sindbis-like plant virus family contains a mutation in the “WFP” motif as depicted at amino acid position 365-367 of SEQ ID NO:2. The vector may be integrated into the genome of said plant cell. The Sindbis-like plant virus is alfalfa mosaic virus, brome mosaic virus, citrus leaf rugose virus, cucumber mosaic virus, sunn-hemp mosaic virus, tobacco mosaic virus, tobacco rattle virus, or turnip vein clearing virus. The invention further embodies a plant cell transformed according to this method and the plant generated from the transformed plant cell.

In another aspect, the invention is furthermore a purified nucleic acid comprising a nucleic acid fragment encoding a membrane binding protein from the Sindbis-like plant virus fused to a DNA sequence encoding a protein of interest. The Sindbis-like plant virus is alfalfa mosaic virus, brome mosaic virus, citrus leaf rugose virus, cucumber mosaic virus, sunn-hemp mosaic virus, tobacco mosaic virus, tobacco rattle virus, and turnip vein clearing virus. The invention is the resulting fusion protein encoded by a purified nucleic acid comprising a nucleic acid fragment encoding a membrane binding protein from the Sindbis-like plant virus fused to a DNA sequence encoding a protein of interest. The resulting fusion protein comprising a membrane binding protein from the Sindbis-like plant virus family containing the “WFP” motif as depicted at amino acid position 365-367 of SEQ ID NO:2 fused to an amino acid sequence of interest has increased stability over the unfused protein of interest expressed in a cell of the same plant species. The invention is also the vector comprising a nucleic acid fragment encoding a membrane binding protein from the Sindbis-like plant virus containing the “WFP” motif as depicted at amino acid position 365-367 of SEQ ID NO:2 fused to a DNA sequence encoding a protein of interest. Additionally, the invention is the plant cell transformed with the vector and the plant generated from the plant cell.

In another aspect, the invention is a purified nucleic acid comprising a nucleic acid fragment encoding a membrane binding protein from the Sindbis-like plant virus containing a mutation in the “WFP” motif as depicted at amino acid position 365-367 of SEQ ID NO:2 fused to a DNA sequence encoding a protein of interest. The Sindbis-like plant virus is alfalfa mosaic virus, brome mosaic virus, citrus leaf rugose virus, cucumber mosaic virus, sunn-hemp mosaic virus, tobacco mosaic virus, tobacco rattle virus, and turnip vein clearing virus. The invention is also the resulting fusion protein encoded by a purified nucleic acid comprising a nucleic acid fragment encoding a membrane binding protein from the Sindbis-like plant virus fused to a DNA sequence encoding a protein of interest. The resulting fusion protein comprising a membrane binding protein from the Sindbis-like plant virus family containing a mutation in the “WFP” motif as depicted at amino acid position 365-367 of SEQ ID NO:2 fused to an amino acid sequence of interest has increased or decreased stability over the unfused protein of interest expressed in a cell of the same plant species. The invention is also the vector comprising a nucleic acid fragment encoding a membrane binding protein from the Sindbis-like plant virus containing a mutation in the “WFP” motif as depicted at amino acid position 365-367 of SEQ ID NO:2 fused to a DNA sequence encoding a protein of interest. Additionally, the invention is the plant cell transformed with the vector and the plant generated from the plant cell.

### DETAILED DESCRIPTION

We have identified an amino acid motif, “WFP”, from the TMV 126 kDa and 183 kDa proteins (amino acid position 365 to 367 of SEQ ID:2 and SEQ ID NO:4) that is conserved among viral membrane—associated proteins. The TMV 126 kDa and 183 kDa proteins localize to the ER in infected N. tabacum and N. benthamiana cells. Mutating the “WFP” motif to “WYP” or “WAP” resulted in a variety of effects, somewhat dependent upon the host species. Although the “WFP” motif causes a fused protein to resist ubiquitin-mediated degradation, the mutant 126Y:GFP and 126A:GFP resulted in an increased degradation of a fused protein. Thus, we disclose a method to modulate the rate, and therefore the stability, of an engineered protein.

One method to decrease the rate of degradation of an engineered protein in plant cells includes creating a vector expressible in a plant cell, wherein the vector encodes a fusion protein between the TMV 126 kDa protein and a protein of interest. An exemplary nucleotide sequence for inclusion in this vector is SEQ ID NO:1 which encodes the TMV 126 kDa protein of SEQ ID NO:2. The vector could be designed for transient transfection, or for integration into the plant cell's genome. After creating the vector expressible in a plant cell, the method includes introducing the vector into one or more plant cells through any currently known methods of the art or other methods that will be known. The resulting plant cell containing the vector expresses the fusion protein, which has a decreased rate of degradation compared to the protein of interest when not expressed as a fusion protein. In addition to the method described for decreasing the rate of degradation of a protein of interest, the invention as disclosed herein also includes the vector created for implementing the disclosed method, the nucleotide sequence that encodes the fusion protein, the fusion protein that results from the expression of the created vector, the plant cell or cells transformed with the created vector, and the plants that are generated from the transformed cells.

Another method to decrease the rate of degradation of an engineered protein in plant cells includes creating a vector expressible in a plant cell, wherein the vector encodes a fusion protein between the TMV 183 kDa protein and a protein of interest. An exemplary nucleotide sequence for inclusion in this vector is SEQ ID NO:3 which encodes the TMV 186 kDa protein of SEQ ID NO:4. The vector could be designed for transient transfection, or for integration into the plant cell's genome. After creating the vector expressible in a plant cell, the method includes introducing the vector into one or more plant cells through any currently known methods of the art or other methods that will be known. The resulting plant cell containing the vector expresses the fusion protein, which has a decreased rate of degradation compared to the protein of interest when not expressed as a fusion protein. In addition to the method described for decreasing the rate of degradation of a protein of interest, the invention as disclosed herein also includes the vector created for implementing the disclosed method, the nucleotide sequence that encodes the fusion protein, the fusion protein that results from the expression of the created vector, the plant cell or cells transformed with the created vector, and the plants that are generated from the transformed cells.

The invention also includes methods to increase the rate of degradation of an engineered protein in plant cells. This method includes creating a vector expressible in a plant cell, wherein the vector encodes a fusion protein between a mutant TMV 126 kDa protein and a protein of interest. An exemplary nucleotide sequence for inclusion in this vector is SEQ ID NO:5 which encodes a mutant TMV 126 kDa protein of SEQ ID NO:6 where amino acid 366 is any amino acid but phenylalanine. Two exemplary amino acid substitutions include tyrosine and alanine. The vector could be designed for transient transfection, or for integration into the plant cell's genome. After creating the vector expressible in a plant cell, the method includes introducing the vector into one or more plant cells through any currently known methods of the art or other methods that will be known. The resulting plant cell containing the vector expresses the fusion protein, which has an increased rate of degradation compared to the protein of interest when not expressed as a fusion protein. In addition to the method described for increasing the rate of degradation of a protein of interest, the invention as disclosed herein also includes the vector created for implementing the disclosed method, the nucleotide sequence that encodes the fusion protein, the fusion protein that results from the expression of the created vector, the plant cell or cells transformed with the created vector, and the plants that are generated from the transformed cells.

The invention also includes methods to increase the degradation rate of an engineered protein in plant cells. This method includes creating a vector expressible in a plant cell, wherein the vector encodes a fusion protein between a mutant TMV 183 kDa protein and a protein of interest. An exemplary nucleotide sequence for inclusion in this vector is SEQ ID NO:7 which encodes a mutant TMV 183 kDa protein of SEQ ID NO:8 where amino acid 366 is any amino acid but phenylalanine. Two exemplary amino acid substitutions include tyrosine and alanine. The vector could be designed for transient transfection, or for integration into the plant cell's genome. After creating the vector expressible in a plant cell, the method includes introducing the vector into one or more plant cells through any currently known methods of the art or other methods that will be known. The resulting plant cell containing the vector expresses the fusion protein, which has an increased rate of degradation compared to the protein of interest when not expressed as a fusion protein. In addition to the method described for increasing the degradation rate of a protein of interest, the invention as disclosed herein also includes the vector created for implementing the disclosed method, the nucleotide sequence that encodes the fusion protein, the fusion protein that results from the expression of the created vector, the plant cell or cells transformed with the created vector, and the plants that are generated from the transformed cells.

As anyone skilled in the art can recognize, other nucleotide sequences that encode amino acid sequences with analogous function and homologous sequence to TMV's 126/183 kDa protein may be used to decrease the degradation rate of an engineered protein. This method to decrease the degradation rate of an engineered protein in plant cells includes creating a vector expressible in a plant cell, wherein the vector encodes a fusion protein between a protein with analogous function and homologous sequence to TMV's 126/183 kDa protein from one of the following Sindbis-like plant viruses: alfalfa mosaic virus, brome mosaic virus, citrus leaf rugose virus, cucumber mosaic virus, sunn-hemp mosaic virus, tobacco mosaic virus, tobacco rattle virus, and turnip vein clearing virus. The vector could be designed for transient transfection, or for integration into the plant cell's genome. After creating the vector expressible in a plant cell, the method includes introducing the vector into one or more plant cells through any currently known methods of the art or other methods that will be known. The resulting plant cell containing the vector expresses the fusion protein, which has a decreased degradation rate compared to the protein of interest when not expressed as a fusion protein. In addition to the method described for decreasing the degradation rate of a protein of interest, the invention as disclosed herein also includes the vector created for implementing the disclosed method, the nucleotide sequence that encodes the fusion protein, the fusion protein that results from the expression of the created vector, the plant cell or cells transformed with the created vector, and the plants that are generated from the transformed cells.

Yet another method to increase the degradation rate of an engineered protein in plant cells includes creating a vector expressible in a plant cell, wherein the vector encodes a fusion protein between a mutated protein with analogous function and homologous sequence to TMV's 126/183 kDa protein from one of the following Sindbis-like plant viruses: alfalfa mosaic virus, brome mosaic virus, citrus leaf rugose virus, cucumber mosaic virus, sunn-hemp mosaic virus, tobacco mosaic virus, tobacco rattle virus, and turnip vein clearing virus. The vector could be designed for transient transfection, or for integration into the plant cell's genome. After creating the vector expressible in a plant cell, the method includes introducing the vector into one or more plant cells through any currently known methods of the art or other methods that will be known. The resulting plant cell containing the vector expresses the fusion protein, which has an increased degradation rate compared to the protein of interest when not expressed as a fusion protein. In addition to the method described for increasing the degradation rate of a protein of interest, the invention as disclosed herein also includes the vector created for implementing the disclosed method, the nucleotide sequence that encodes the fusion protein, the fusion protein that results from the expression of the created vector, the plant cell or cells transformed with the created vector, and the plants that are generated from the transformed cells.

Materials and Methods

Plant Materials

Nicotiana benthamiana and Nicotiana tabacum Xanthi “nn” and “NN” were germinated in a tray and individually transplanted into 12 cm pots containing an artificial soil medium (Metro-Mix 350, Grace). Plants were grown in the greenhouse until needed under the following conditions: 16 hour and 25° C. days and 8 hour and 17° C. nights. Supplemental light intensity was 500 μmol photons M⁻² s⁻¹. Plants used for inoculation experiments were six to seven weeks old. Although other conditions may be used, the above growth conditions are preferred.

Suspension Cells, Protoplasts and Transfection

The maintenance of suspension cells, preparation of protoplasts and transfection of protoplasts by electroporation were conducted according to Watanabe et al.(1987), modified for electroporating the N. benthamiana cells and protoplasts. N. tabacum BY-2 (Dr. Richard Cyr, Penn State University) suspension cells were grown in 50 ml of culture media (4.3 g/L M&S salt, 100 mg/L myo-inositol, 1 mg/L thiamine, 0.2 mg/L 2,4-D, 255 mg/L KH₂PO₄, 30 g/L sucrose, pH 5.0) at 26° C. constantly shaking at 150 rpm and sub-cultured weekly. Suspension cells of N. benthamiana (Dr. Bryce Falk, University

California—Davis) were grown in culture media (4.3 g/L M&S salt, 0.204 g/L KH₂PO₄, 100 mg/L myo-inositol, 0.2 mg/L 2,4-D, 0.1 mg/L Kinetin, 1 mg/L thiamine, 0.5 mg/L pyridoxide, 0.5 mg/L nicotinic acid, 30 g/L sucrose, pH 5.8) at 26° C. constantly shaking at 150 rpm and sub-cultured every 10 days.

In order to create protoplasts, both BY-2 and N. benthamiana cells were digested with 1% Cellulose, R-10; 0.1% Pectolyase Y-23 and 1% Driselase (Karlan) in MMC buffer (13% mannitol, 5 mM MES, 10 mM CaCl₂, pH 5.8) at room temperature for 3 hours. The digested cells were overlaid on a 20.5% sucrose cushion and spun at 1,100 rpm on an IEC centrifuge for 11 minutes. The protoplasts on top of the cushion were collected and washed twice with MMC buffer. About 1×10⁶ protoplasts were resuspended in 0.8 ml of the electroporation buffer (13% mannitol, 70 mM KCl, 5 mM MES, pH 5.8).

Fifteen μg of plasmid DNA or 5 μg of in vitro transcript viral RNA (see below) were mixed with 0.8 ml of protoplasts in a precooled cuvette and electroporated with the following setting: 250V, 220 μF and 50 mS (ProGenetor II, Hoefer Scientific Instruments, San Francisco, Calif. USA ). After electroporation, protoplasts were incubated on ice for 10 minutes and washed with 2 ml of MMC buffer. The transfected protoplasts were resuspended in 3 ml of culture media with 13% mannitol and incubated at 26° C. in the dark for BY-2 protoplasts or under light for N. benthamiana protoplasts. Although alternative methods may be employed, the above methods for maintaining suspension cells, creating protoplasts, and transfecting both are preferred.

In Vitro Site-Directed Mutagenesis

To mutate the second amino acid in the “WFP” motif, in vitro site-directed mutagenesis was performed as described before (Bao et al, 1996). The phenylalanine in the “WFP” motif from M^{IC}m2 (an infectious transcript of M^{IC} TMV altered at a single nucleotide to the UI strain sequence in the 126 kDa protein open reading frame) (Shintaku et al., 1996) was replaced with alanine and tyrosine, respectively. In order to create the “WAP” motif, in vitro site-directed mutagenesis was performed using the following primer complementary to nucleotides 1141-1177: 5′-CTCATTTCGGGAGCCCAGTAATTGACTGATGATGAAT-3′ (SEQ ID NO:22). In order to create the “WYP” motif, in vitro site-directed mutagenesis was performed using the following primer complementary to nucleotides 1141-1173: 5′-TTTCGGGATACCAGTAATTGACTGATGATGAAT-3′ (SEQ ID NO:23). The underlined codon indicates the mutated sites. All mutant clones were confirmed to contain the specified alteration by sequence analysis. Although site-directed mutagenesis to the WAP and WYP motifs may be performed using alternative primers, the above methods are preferred. Additionally, other mutations can be made in the place of phenylalanine 366 as numbered in SEQ ID NO:2 in the same way.

In Vitro Transcription and Inoculation

Plasmid DNA of infectious TMV cDNA clones was linearized by Acc65 I and gel-purified to act as a template in the in vitro transcription reaction performed as described previously (Shintaku et al., 1996). 5 μg of transcript viral RNA was inoculated on the mature leaves of N. benthamiana, N. tabacum Xanthi “NN”, and “nn” which were dusted with the abrasive carborundum. The inoculated plants were kept in the greenhouse to observe local lesions and systemic symptoms. Other method may be utilized for in vitro transcription and inoculation, but the processes described above are preferred.

Construction of 126 kDa-GFP Fusion Chimeric Vectors

A cDNA fragment encoding the 126 kDa protein of the M^{IC} TMV was amplified from plasmid L19 (Shintaku et al., 1996) using the Pfu polymerase (Stratagene) and a pair of primers ST (5′-CCATGCCATGGCGCTCGAGATGGCATACACACAGACA-3′ (SEQ ID NO:24), where the underlined nucleotides indicate the TMV genome sequence from the position 69 to 86) and GT (5′-CCCTTGCTCACCATTTGTGTTCCTGCATCG-3′ (SEQ ID NO:25), where the underlined nucleotides indicate the sequence complementary to TMV genome sequence, from the position 3401 to 3416). Green fluorescent protein (GFP) (EGFP, Clontech Laboratories, Inc., Palo Alto, Calif.) was amplified from plasmid pEGFP (Clontech) using the Pfu polymerase and a pair of primers TG (5′-ATGCAGGAACACAAATGGTGAGCAAGGGCG-3′) (SEQ ID NO:26) and 3GFP (5′-CCATGCCATGGCTCGAGTTACTTGTACAGCTCGT-3′) (SEQ ID NO:27). The amplified fragments were gel-purified and mixed as the template for the fusion PCR using the primers 5T and 3GFP (method described by Higuchi, 1990). The PCR product was the fusion of the 126 kDa protein gene and the GFP gene which was purified and digested with Nco I. The digested fragment was purified and ligated with plasmid pRTL2 (Restrepo et al., 1990) previously digested with Nco I. The ligation mixture was transformed into E. coli HB 101. The clone containing the insert having the correct orientation was identified by restriction digestion and sequencing, and named p126:GFP. To make the mutated 126K fusion protein construct, the infectious cDNA clones of “WFP”, “WYP” and “WAP” were digested with Mlu I and Dra III, sequentially. The Mlu I-Dra III fragments from each of the clones were inserted into the same site of p126:GFP previously digested with Mlu I and Dra III. Those clones containing wild type “WFP” motif and the mutated motifs (“WYP” and “WAP”) were named p126F:GFP, p126Y:GFP and p126A:GFP, respectively. Although a variety of methods could be utilized to create chimeric vectors, the above methods are preferred. Although only the full length 126 kDa protein was fused to a gene of interest, this application anticipates that truncated portions of the TMV 126 kDa protein or peptides can also be employed in the present invention as long as the amino acid sequence that stabilizes the fusion protein contains the “WFP” motif or elements that act in the same fashion.

Biolistic Bombardment and Fluorescent Microscopy

Transient expression of 126 kDa-GFP fusion protein in tobacco leaves by biolistic bombardment was performed according to Itaya, et al. (1997). Five μg of each of p126F:GFP, p126Y:GFP and p126A:GFP was bombarded into the lower epidermis of N. benthamiana and N. tabacum Xanthi nn leaves using a Biolistic PDS 1000/He System (Bio-Rad) at a pressure of 1,100 psi. The bombarded leaves were incubated in a sealed petri dish with several pieces of water-soaked filter paper at 25° C. with light overnight.

The leaves were observed under a Nikon Microphot-FX epifluorescent microscope with a filter set B-2A, consisting of a blue excitation filter (450-490 nm), a dichroic mirror (510 nm) and a barrier filter (520 nm). Fluorescent images were photographed with the camera system attached to the microscope using Kodak Royal 400 color film. While biolistic bombardment and fluorescent microscopy could be accomplished in different ways, the above methods are preferred.

Transient Expression of 126F:GFP, 126Y:GFP and 126A:GFP in Protoplasts

Fifteen μg of plasmid DNA of the three fusion protein constructs (126F:GFP, 126Y:GFP and 126A:GFP) were transfected into protoplasts of N. benthamiana and BY-2 cells by electroporation as described above. The transfected protoplasts were collected at 7, 12, 16, 18, 24, 36, 48, 72, and 96 hours post-incubation and plated on a 12-well slide for a single cell time course observation with a procedure as described previously (Mas and Beachy, 1998). The fluorescent fusion protein expression in the protoplasts was examined by confocal laser scanning microscopy (CLSM) as described below.

Immunofluorescent Labeling

Immunofluorescent labeling of TMV 126K protein and host components was conducted according to Heinlein et al. (1995) with a minor modification as follows. First, 0.5 ml of protoplasts of N. benthamiana and BY-2 infected with “WFP”, “WYP” and “WAP” viruses were harvested 2 days post-infection. The protoplasts were spun down at 700 rpm in 14 ml tubes (Falcon) at room temperature for 2 minutes and resuspended in fixative buffer (50 mM Na₂HPO₄, pH 6.7; 4% paraformadehyde, 0.1% glutaradehyde, 5 mM EGTA, pH 8.0) for 30 minutes at room temperature. The fixed protoplasts were plated on the slides precoated with 0.1% poly-L-lysine and then extracted with cold methanol for 10 minutes. All washes were performed in phosphate-buffered saline (PBS), pH 7.0, containing 0.5% Tween-20 and 5 mM EGTA. Primary antibodies were polyclonal rabbit IgG recognizing the TMV 126K protein (Nelson, et al. 1993) and polyclonal rabbit IgG against BiP, an ER associated protein indicator, kindly provided by Dr. Becky Boston, North Carolina State University. Secondary antibodies were FITC-conjugated goat anti-rabbit IgG and Texas Red-conjugated goat anti-mouse IgG (Molecular Probes, Eugene, Oreg., USA). The samples were mounted with mounting media ( 0.1 M Tris-HCl, pH 9.0; 50% glycerol, 1 mg/ml p-phenylenediamine) and stored at 4° C. before observation. Other methods and materials may be used to visualize fusion protein presence and localization, but the above methods and materials are preferred.

Proteosome Inhibition

ALLN (N-acetyl-L-leucinyl-L-leucinyl-L-norleucinal, Sigma Chemical Co. St. Louis, Mo.) was used at a final concentration of 75 μM in dimethyl sulfoxide (DMSO). The BY-2 protoplasts transfected with fusion protein constructs were incubated in the culture media containing 75 μM of ALLN and collected 12, 24, and 48 hours post-transfection. The transient fluorescent protein expression in protoplasts was examined by CLSM as described below. There may be other ways to perform the inhibitor experiment, but the above methods are merely preferred.

Confocal Microscopy

Immnunofluorescent labeling signals and transient expression of 126 kDa:GFP fusion protein in protoplasts were examined with CLSM (Cheng et al., 2000). Most images were captured with 3% laser power, but in the inhibitor experiment, 10% laser power was used. The above conditions are merely representative of conditions used to visualize data with confocal microscopy.

### EXAMPLE 1

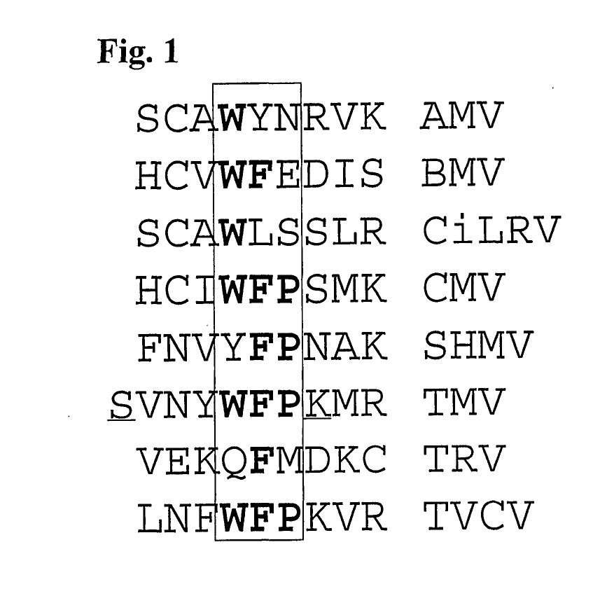

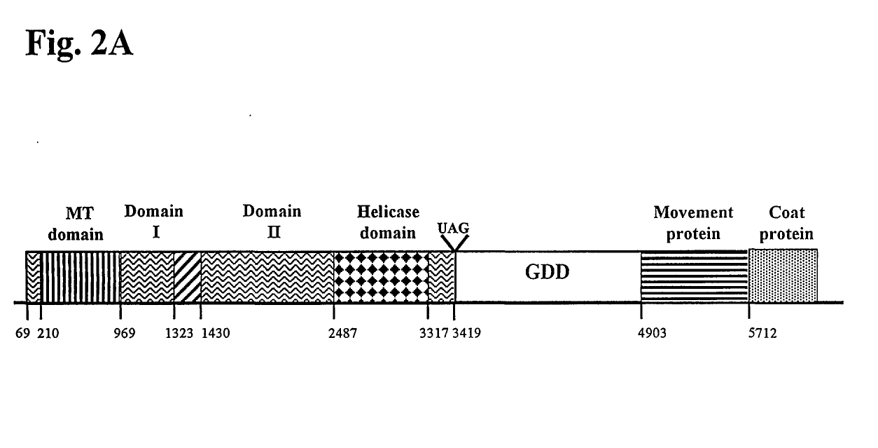

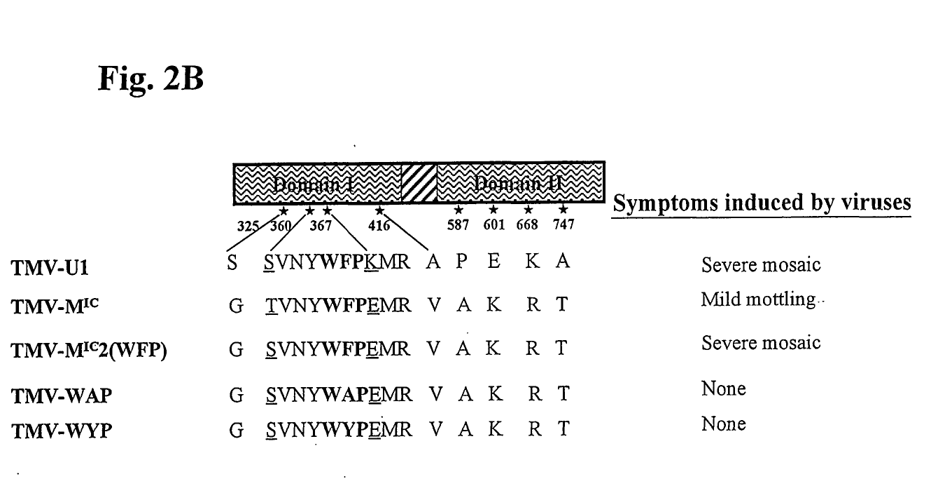

To better understand how the domains within the TMV 126 kDa protein influence pathophysiology, the sequence of the TMV 126 kDa protein was compared to functionally related proteins from other Sindbis-like plant viruses: alfalfa mosaic virus, brome mosaic virus, citrus leaf rugose virus, cucumber mosaic virus, sunn-hemp mosaic virus, tobacco rattle virus, and turnip vein clearing virus. The TMV 126 kDa protein was aligned with its functional analogues from other Sindbis-like plant viruses using the CLUSTAL W program (Thompson et al., 1994) to identify a conserved “WFP” sequence (trypotophan-phenylalanine-proline) (FIG. 1). The “WFP” sequence is contained within Domain I, between the methyltransferase and helicase domains of this protein (FIG. 2A). This “WFP” sequence was also found in several plant proteins, most of which are membrane-associated. A person skilled in the art, understanding concepts of amino acid homology and functionally analogous proteins, will also recognize that the alignment of FIG. 1 identifies parts of other sequences that may be fused to stabilize an engineered protein. Like the TMV 126 kDa protein used herein, some of the proteins in FIG. 1 have a putative ER-colocalizing signal that may be mutated to destabilize a fused engineered protein.

To create a destabilizing motif, three mutant viruses were constructed that were altered within this motif (FIG. 2B). The WFP virus refers to a virus with a masked (M^{IC}) genetic background, except for a “Ser” residue, found in the U1 strain, at position 325 (Shintaku et al., 1996). This sequence alteration results in the WFP virus (also referred to as M^{IC} m²) inducing severe symptoms and accumulating more efficiently in systemic tissue than the parental M^{IC} virus (Derrick et al., 1997). The WAP and WYP viruses were constructed by replacing “Phe” with “Ala” or “Tyr”, respectively, of the 126 kDa protein (FIG. 2B). Both mutations of the “WFP” motif resulted in a virus unable to cause symptoms of the parental Tobacco mosaic virus. Although only alanine and tyrosine were substituted for phenylalanine in this present example, any substitute amino acid not having phenylalanine characteristics is anticipated in this invention because it acts to destabilize the fused protein.

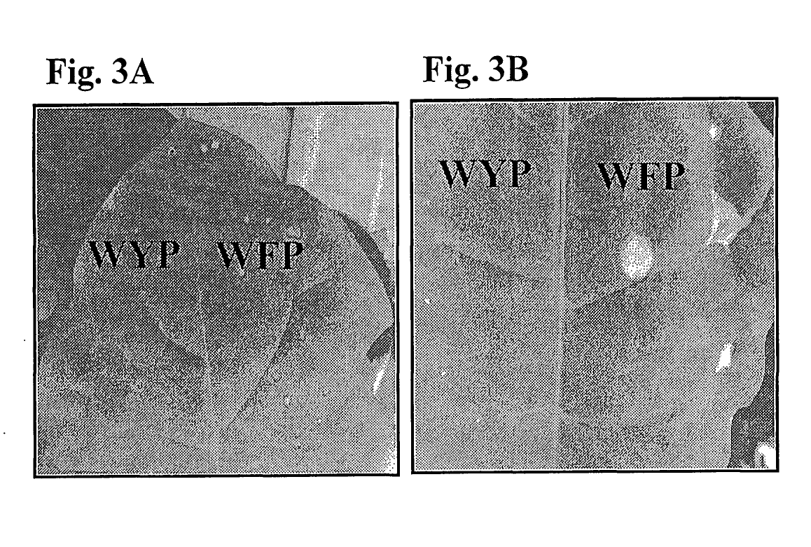

Changing the phenylalanine to either alanine or tyrosine in the WFP motif decreased the infectivity of the mutant viruses on tobacco species. The WAP virus did not infect N. tabacum plants, but did infect N. benthamiana plants (Table 1). The WYP virus induced only mild systemic symptoms on N. tabacum plants but severe systemic symptoms on N. benthamiana. The wild-type WFP virus induced severe symptoms on both Nicotiana species (Table 1). On N. tabacum Xanthi “NN” plants, a local lesion host for TMV, the WYP virus induced tiny necrotic lesions at 24° C., whereas the WFP virus induced larger lesions (FIG. 3A). High temperature treatment of 32° C. for three days before returning to 24° C. blocked the necrotic response of Nicotiana, but did not affect the lesion size induced by the WYP virus on “NN” plants (FIG. 3B). The WFP virus, however, induced larger lesions after returning to the lower temperature (FIG. 3B). These data demonstrate that the “WFP” motif within the 126 kDa protein is required for efficient virus replication and infection, and that the necrosis response does not limit the infectivity of the WYP virus.

TABLE 1
Summary of biological analyses of the WFP, WYP, and WAP
viruses in Nicotiana tabacum and Nicotiana benthamiana

| Host | Phenotypes | WFP | WAP | WYP |
|---|---|---|---|---|
| N. tabacum | Replication | + | − | + |
| N. tabacum | Cell to cell | + | − | + |
| | movement | | | |
| N. tabacum | Systemic | severe | none | mild^{a} |
| | symptoms | | | |
| N. benthamiana | Replication | + | − | + |
| N. benthamiana | Cell to cell | + | − | + |
| | movement | | | |
| N. benthamiana | Systemic | very severe | mild | severe^{b} |
| | symptoms | | | |

^{a}Often due to second site mutations occurring in progeny virus.
^{b}Not due to second site mutations occurring in progeny virus.

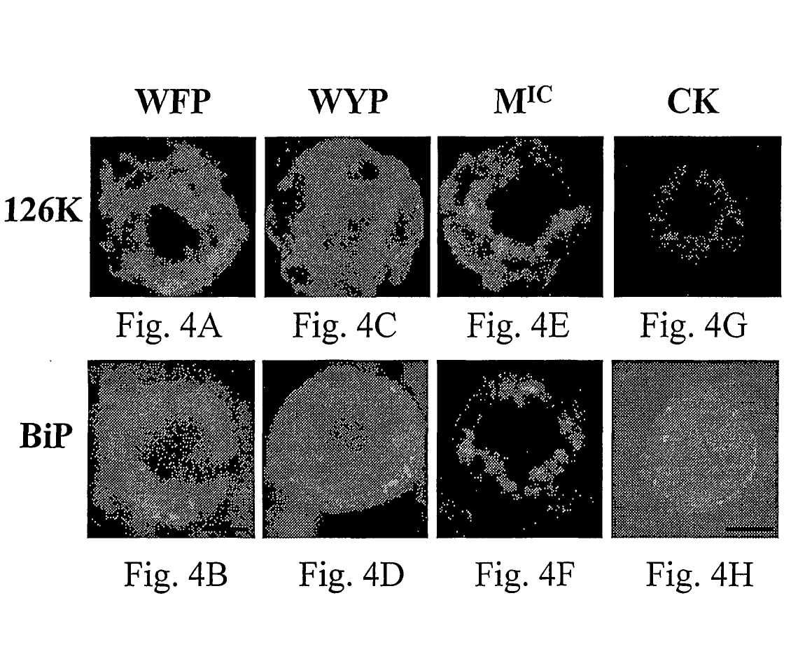

We immunolabeled N. tabacum (cv. BY-2) and infected with the WFP, WYP or WAP viruses using antibodies against the TMV 126 kDa and binding protein (BiP), an ER marker (FIGS. 4A-H). The TMV 126 kDa protein containing the “WFP” motif (both the WFP and M^{IC} viruses) localized to subcellular bodies similar to those observed in cells probed with anti-BiP (FIGS. 4A, 4B, 4E, and 4F). Both the 126 kDa protein containing the “WYP” motif (FIG. 4C) and BiP (FIG. 4D) failed to localize in N. tabacum cells inoculated with WYP virus. Interestingly, the TMV 126 kDa protein was not detected at all in WAP virus-infected cells of N. tabacum. There was no TMV 126 kDa protein detected in the mock-infected N. tabacum protoplast (FIG. 4G). In N. benthamiana protoplasts, the 126 kDa proteins of the WYP and WAP viruses localized similarly to the 126 kDa protein from the WFP virus (data not shown). These results indicate that the “WFP” motif within the TMV 126 kDa protein is necessary for the proper interaction of the TMV 126 kDa protein with host factors to localize to the ER, and this association is correlated with the ability of the virus to efficiently infect the host. Altering the “WFP” motif prevents localization to the ER.

### EXAMPLE 2

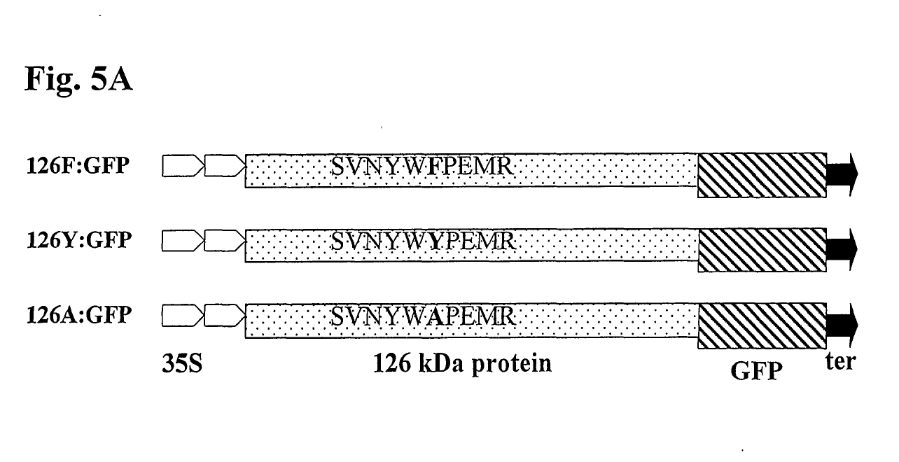

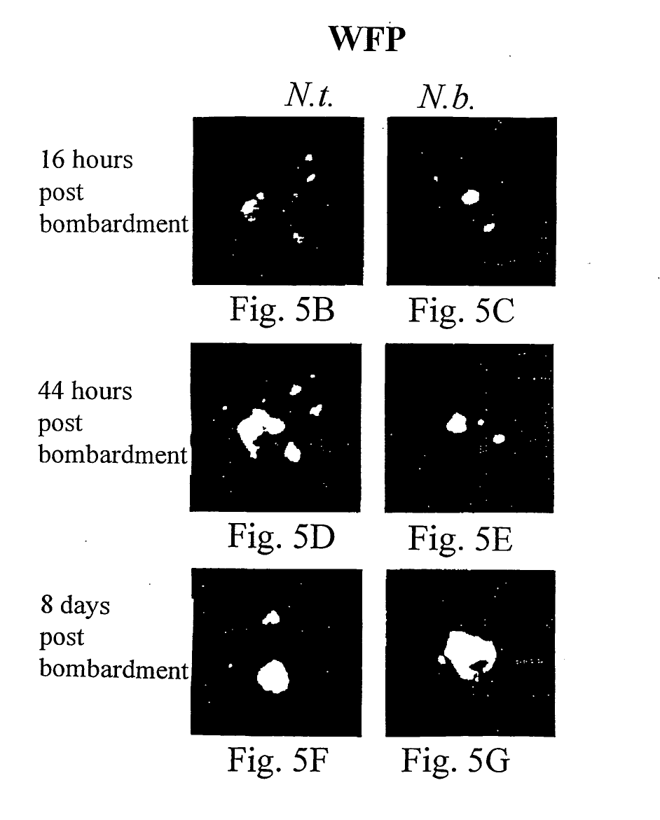

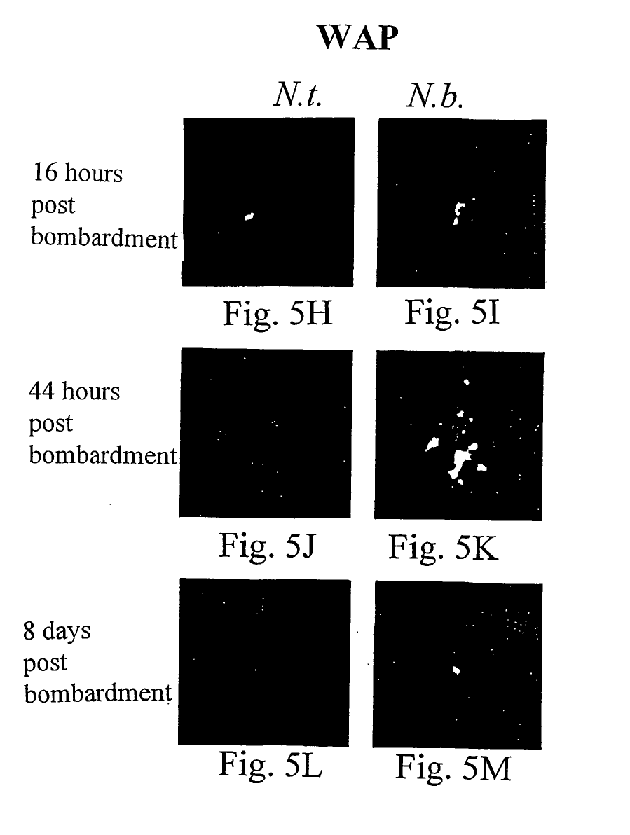

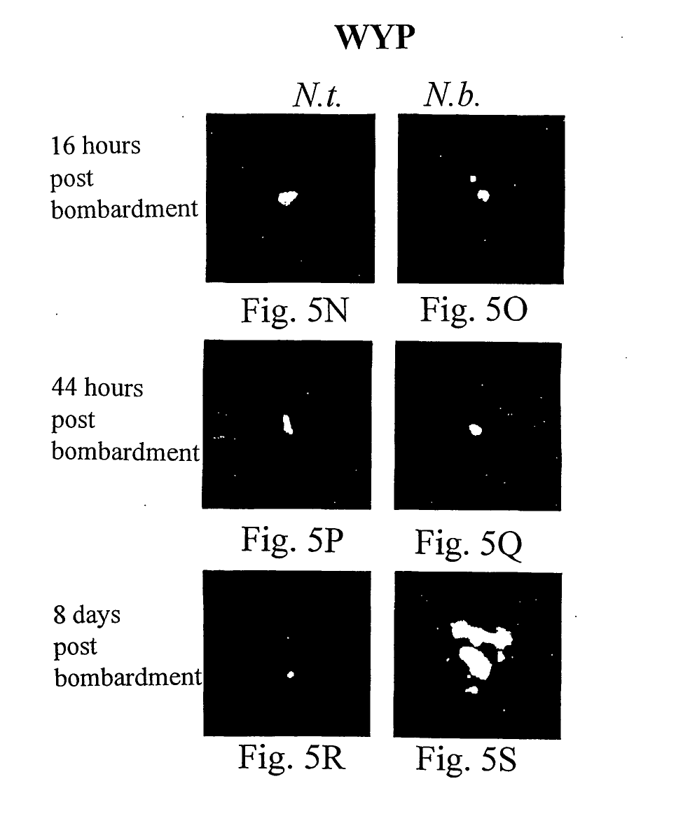

The TMV 126 kDa protein ORFs from the “WFP”, “WYP”, and “WAP” viruses were fused with GFP ORF to yield 126F:GFP (containing the “WFP” motif), 126Y:GFP (containing the “WYP” motif) and 126A:GFP (containing the “WAP” motif) constructs. These constructs were placed behind an enhanced 35S promoter for transient expression in both N. tabacum Xanthi nn and N. benthamiana leaf cells by biolistic bombardment (FIG. 5A). The fluorescent signal was observed in subcellular bodies as punctate dots and along the periphery of the cells (FIGS. 5B-5S). The fluorescent 126F:GFP was stable for at least 8 days in both Nicotiana species (FIGS. 5B-5G), while the intensity of fluorescence declined rapidly for the 126A:GFP and 126Y:GFP fusions in N. tabacum (FIG. 5H, 5J, 5L, 5N, 5P, and 5R). In N. benthamiana, however, the fluorescence produced by the 126Y:GFP fusion was not reduced relative to the 126F:GFP fusion over time (FIGS. 5S and 5G). The stability pattern of the various transiently expressed 126 kDa:GFP fusion proteins correlated with the ability of the parental and mutant viruses to efficiently infect the host. This finding also shows that the stabilization of viral replicase complex through the altered 126 kDa protein requires species-specific host factors.

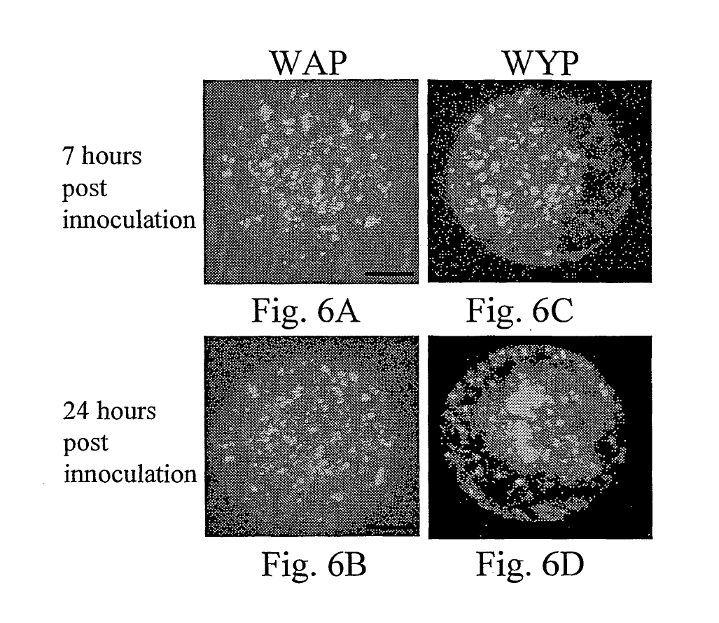

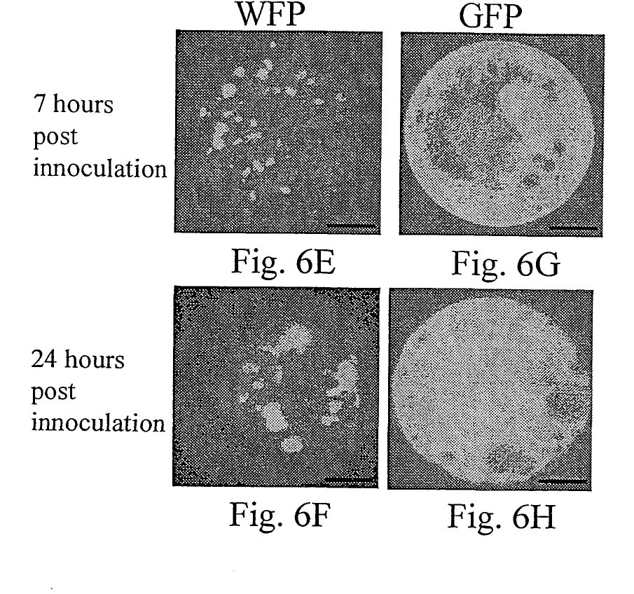

N. benthamiana protoplasts were transfected with 126F:GFP-, 126Y:GFP-, and 126A:GFP-containing plasmids to study the subcellular localization of the 126 kDa:GFP fusion proteins during transient expression. The fusion proteins formed many small irregular bodies within the cytosol (FIGS. 6A, 6C, and 6E), unlike the non-fused GFP construct which failed to form subcellular bodies 7 hours post-inoculation (FIG. 6G). At 24 hours after inoculation, the protoplasts expressing the 126F:GFP and 126Y:GFP constructs appeared to have fewer, but larger fluorescent bodies (FIGS. 6D and 6F). The protoplasts expressing free GFP formed no punctate bodies even after 24 hours (FIGS. 6G and 6H).

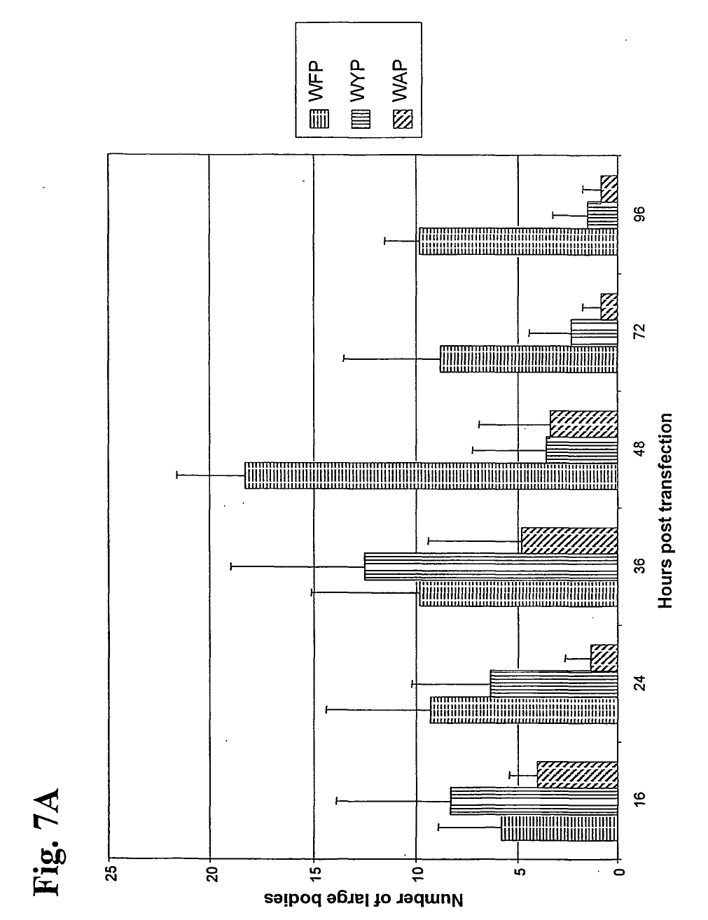

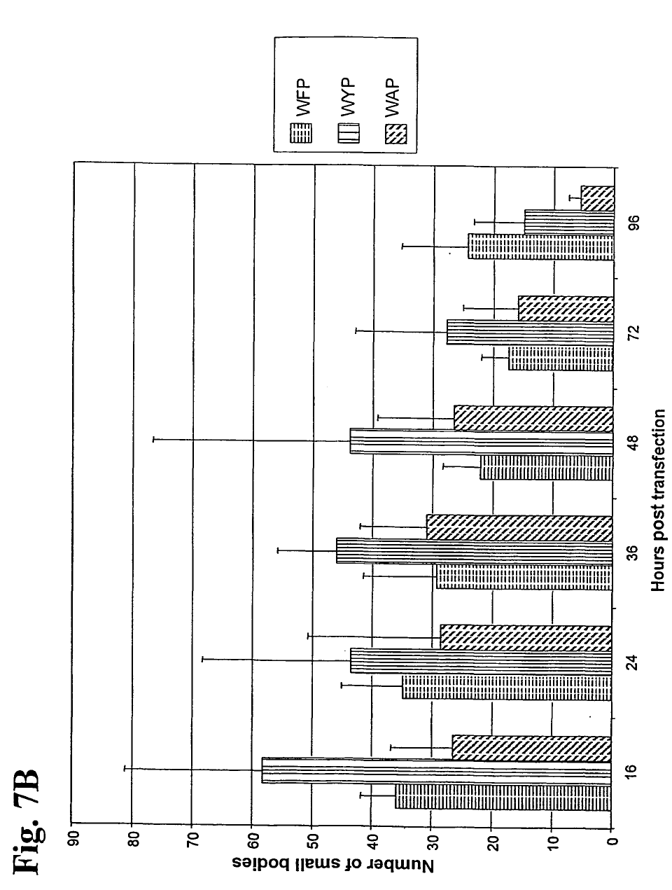

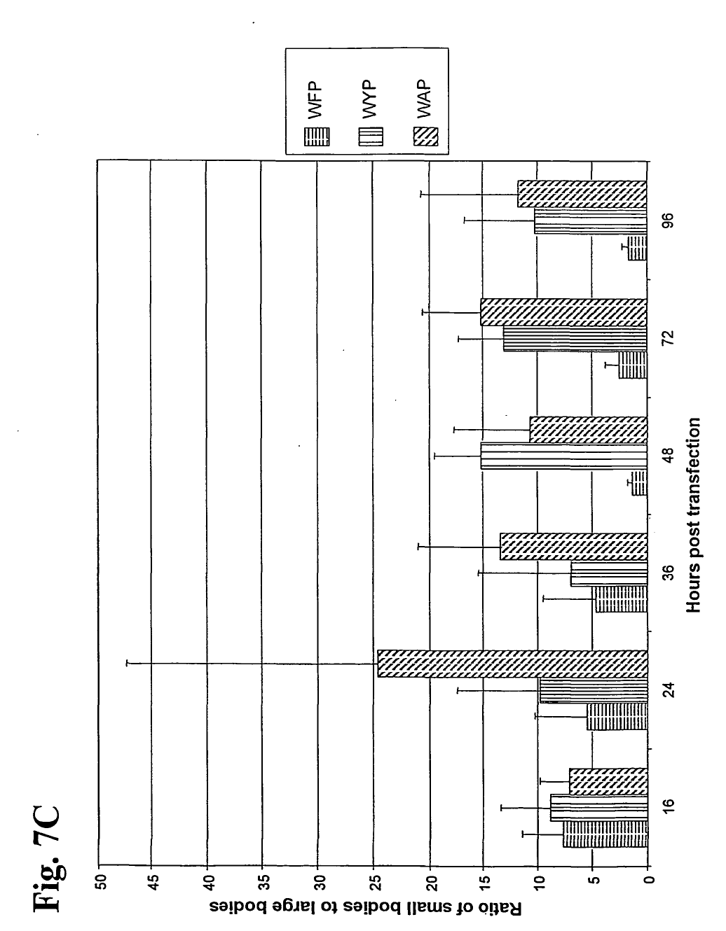

N. tabacum (cv. BY-2) protoplasts were also transfected with 126F:GFP-, 126Y:GFP-, and 126A:GFP-containing plasmids. The irregular fluorescent bodies that resulted could be categorized into two types: small bodies less than 2 μm in diameter which disappeared over time, and large bodies more than 2 μm in diameter which persisted. The wild-type 126F:GFP fusion protein formed both types of bodies in BY-2 cells (FIGS. 7A and 7B). The 126Y:GFP and 126A:GFP fusion proteins formed mostly only small bodies (FIG. 7B). Generally, the 126A:GFP fusion protein produced fewer large bodies than did the 126Y:GFP fusion protein (FIG. 7A). Also, the small bodies produced by the 126A:GFP fusion protein disappeared even more rapidly than did those formed by the 126Y:GFP fusion protein (FIG. 7A). These results indicated that the 126 kDa protein alone, even without other viral proteins, localized to the ER in infected cells. A determinant that controls localization of TMV 126 kDa protein to the ER is the “WFP” motif or the motif affected by the “WFP” motif.

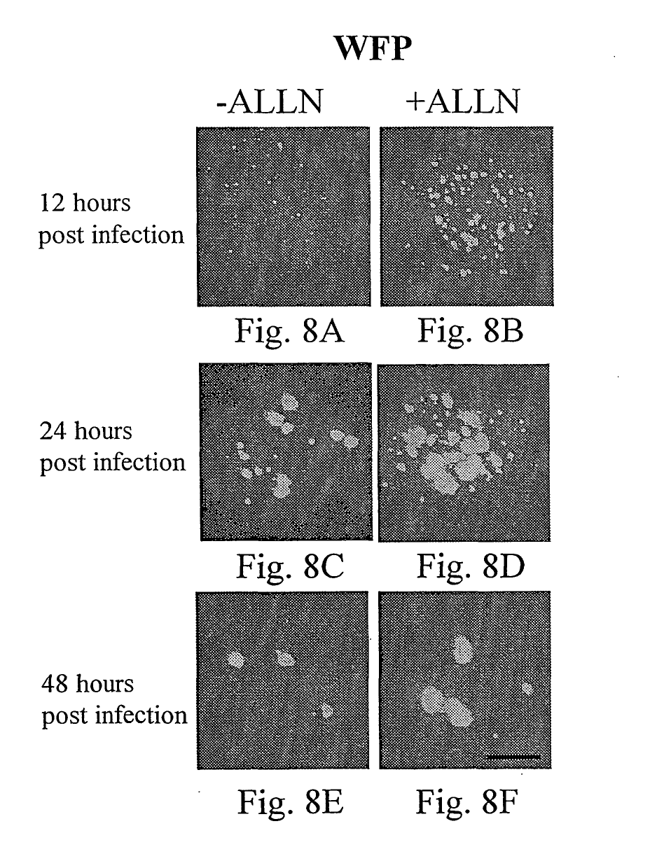

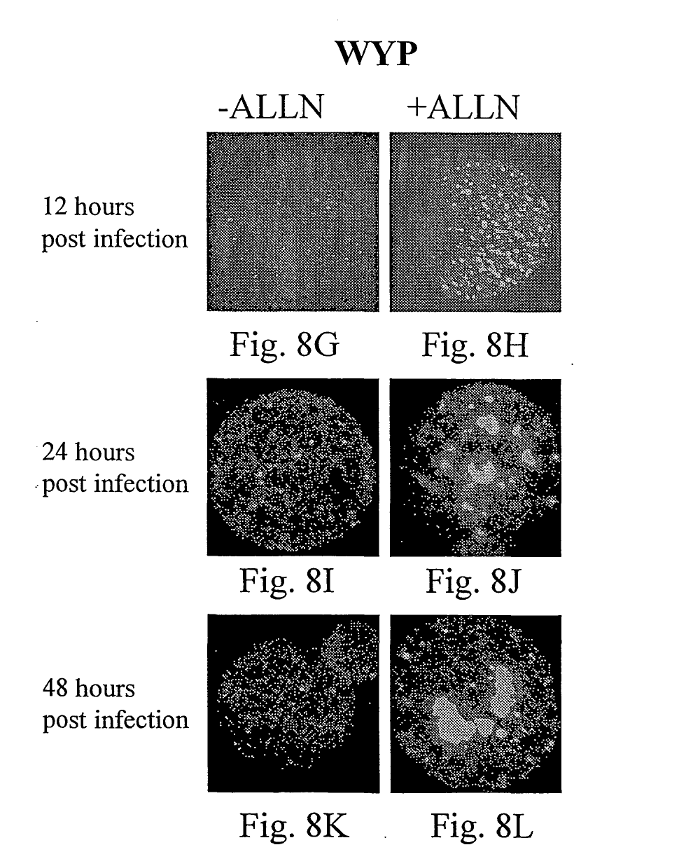

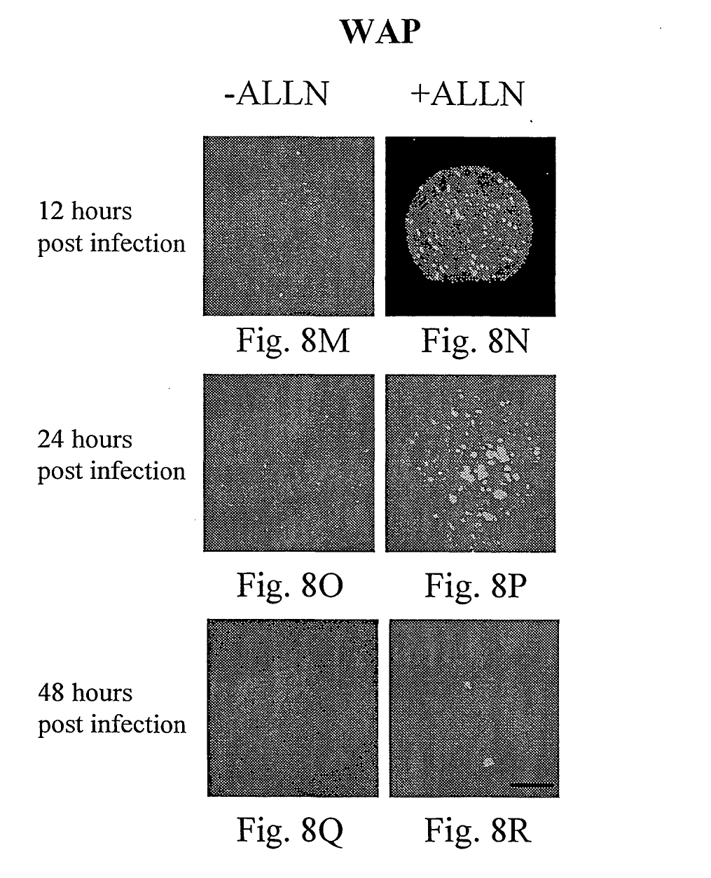

The previous results indicated that the altered 126 kDa:GFP fusion proteins were less stable than the “WFP” containing fusion protein in BY-2 cells. To determine if the 26S proteosome was responsible for degrading these TMV proteins, we expressed the fusion proteins in BY-2 cells incubated in the presence or absence of Acetyl-Leu-Leu-norleucinal (ALLN), an inhibitor of the 26S proteasome. Cells incubated in ALLN and transfected with either of the mutant 126Y:GFP or 126A:GFP fusion constructs yielded fluorescent signals that were greater and more stable compared to the signals from transfected cells without ALLN (compare FIGS. 8G, 8I, 8K, 8M, 8O, and 8Q to FIGS. 8B, 8D, 8F, 8H, 8J, and 8L). In the ALLN-treated cells, the 126Y:GFP fusion protein produced more fluorescent small bodies and also formed the large irregular bodies that localized around the nucleus at late stages, similar to what was observed for the 126F:GFP fusion protein (FIGS. 8G-8L for 126Y:GFP and compare to FIGS. 8B, 8D, and 8E for 126F:GFP). This result demonstrates that the “WYP” fusion protein can form small bodies in the absence of ALLN, but cannot avoid the host degradation machinery in the absence of inhibitor, thereby leading to an inability to form the large stable bodies. Also, the presence of the inhibitor led to greater expression of the 126F: wild-type GFP fusion than in its absence (FIG. 8B, 8D, 8F, versus 8A, 8C, and 8E). These findings indicate that the instability of the altered 126 kDa:GFP fusion proteins was due to their degradation by the host 26S proteasome. The maintenance of the “WFP” motif within the 126 kDa protein was thus critical to inhibit the degradation of this protein by the host ubiquitin-facilitated pathway. The ability of the altered viral proteins to form bodies in N. benthamiana cells and not in N. tabacum BY2 cells showed that the ability to degrade the viral protein is controlled by host factors in N. tabacum that better recognize structural change in the target than those from N. benthamiana. Therefore, protein with the WFP motif resists ubiquitin-dependent degradation.

We have found that the 126 kDa protein stabilizes expression of a fused protein in cells. When the 126 kDa protein was fused with GFP, the expression of the fused protein in the cell cytoplasm, as detected by fluorescence microscopy, was observed for two days longer than unfused GFP. The free GFP was only detectable for up to 5 days, whereas the 126 kDa protein fused with GFP was detectable at 7 days, the last time point collected. Thus, the fusion of the normal 126 kDa protein (i.e. containing the WFP motif) with a foreign protein stabilizes the expression phenotype of the foreign protein.

In summary, an amino acid motif, “WFP”, was identified in the TMV 126 kDa and 183 kDa proteins (amino acid position 365 to 367 as numbered SEQ ID:2 and SEQ ID NO:4) that was conserved among both viral proteins and host membrane-associated proteins. When the “WFP” motif was mutated to “WYP” or “WAP”, the mutant viruses containing these new motifs were dramatically less capable of infecting and replicating in N. tabacum, but could infect N. benthamiana. Immunolabeling of the 126 kDa/183 kDa protein complex in virus-infected cells indicated that the replicase co-localized with binding protein (BiP), a host protein associated with the ER. However, the mutant virus containing WYP failed to localize BiP and the 126 kDa mutant protein to the ER. Transient expression of the 126 kDa protein fused with GFP showed that the mutant 126Y:GFP and 126A:GFP were unstable in plants and protoplasts of N. tabacum, but stable in plants and protoplasts of N. benthamiana. Thus, altering the “WFP” motif resulted in an increased degradation of this fusion protein depending on the host cell species. The wild-type 126 kDa:GFP protein fusions formed cytoplasmic bodies in transfected protoplasts and these bodies could be categorized into two types. Small bodies were less than 2 μm in diameter and disappeared in the WYP- and WAP-transfected cells after 48 hours, and large bodies that were more than 2 μm in diameter that persisted for WFP-transfected cells but not for WYP - or WAP-transfected cells. The 126F:GFP fusion maintained expression of large bodies longer than did 126Y:GFP or 126A:GFP. In the presence of the 26S proteasome inhibitor (ALLN), the 126Y:GFP and 126A:GFP fusions appeared more stable than in the absence of the inhibitor. Thus, the ubiquitin degradation pathway is involved in the degradation of the mutant 126 kDa protein. The accumulation of 126F:GFP fusion protein was increased in the presence of a 26S proteosome inhibitor, indicating some resistance of this protein, even in the absence of other viral proteins, to the ubiquitin degradation pathway.

### EXAMPLE 3

Anyone skilled in the art of protein biochemistry recognizes that the invention herein disclosed may be combined with known methods and materials to yield embodiments not directly mentioned. Because a three amino acid motif within a larger viral ER-colocalizing protein has been identified to render a fused protein more stable in plant cells, a reasonable embodiment of the current invention is to alter the viral ER-colocalizing protein in positions outside the three amino acid motif. By removing portions of the ER-colocalizing protein, it may be possible to minimize the region that confers stability to a fused engineered protein. Alternatively, amino acid substitutions can be made at regions outside the three amino acid motif that confers stability to a fused engineered protein. Naturally, because the truncations and substitutions that will be successful in the invention disclosed are outside the three amino acid motif, they can be used with a mutated the three amino acid motif to render a fused engineered protein unstable.

A person skilled in the art that recognizes the possibility of including truncations and substitutions with the invention described herein will also recognize the possibility of fusing a peptide containing within it the three amino acid motif to a gene of interest to confer stability to the engineered protein. Alternatively, the same peptide when identified may contain a mutated three amino acid motif to render a fused engineered protein unstable.

Literature Cited

Bao et al. 1996 J. Virol. 70: 6378-6383

Bao, Y. and Hull, R. 1993, J Gen Virol 74:1611-1616

Cheng et al., 2000, Plant J. 23: 1-16.

Cheng et al., 2000, Plant J., in press

Deom et al. Science, 1987, 237:389-394

Derrick et al., 1997, Mol. Plant-Microbe Interaction 10: 589-596.

Ecker et al. 1989 J Biol. Chem. 264:7715-779

Heinlein et al. 1995 Science 270: 1983-1985

Heinlein et al., 1998, Plant Cell 10: 1107-1120.

Holt, et al., 1990, MPMI 3:417-423

Higuchi, R. (1990) In “PCR Protocols: A guide to methods and applications” (M. A. Innis, D. H. Gelford, J. J. Sninsky and T. J. White, Eds.) p. 177-183 Academic Press, San Diego

Itaya et al., 1997, Plant J. 12:1223-1230

Janda and Ahlquist 1998, Proc. Natl. Acad. Sci. USA 95: 2227-2232

Lewandowski and Dawson 2000, Virology 271: 90-98.

Laemmli 1970, Nature 227:680-685

Mas and Beachy 1998, Plant J 15:835-842

Mas and Beachy 1999, J. Cell Biol. 147: 945-958.

Nelson, et al. 1993 MPMI 6:45-54

Osman and Buck 1996, J. Virol. 70: 6227-7234.

Reichel and Beachy 2000, J. Virol. 74: 3330-3337.

Restrepo-Hartwig and Ahlquist 1999,J Virol. 73: 10303-10309.

Restrepo-Hartwig et al., 1990 Plant Cell 2:987-998

Shintaku et al., 1996, Virology 221: 218-225.

Sullivan and Ahlquist 1999, J. Virol. 73: 2622-2632

Szecsi et al., 1999, Mol. Plant-Microbe Interaction. 12: 143-152.

Thompson et al., 1994, Nucl. Acids Res. 22: 4673-4680.

Tpfer, et al. 1987, Nucl. Acids Res. 15:5890.

Vierstra, R. D. 1996 Plant Mol. Biol. 32:275-302

Watanabe et al. 1987 FEBS Letters 219:65-69

Watanabe et al., 1999, J. Virol. 73: 2633-2640.

1. A method for decreasing the degradation rate of an engineered protein of interest in a plant cell comprising
constructing a vector comprising a nucleic acid fragment from position 1 to position 3348 of SEQ ID NO:1 fused to a nucleotide sequence encoding said protein of interest, said vector expressible in said plant cell; and
introducing and expressing said vector in said plant cell to form a fused protein;
wherein the degradation rate of said fused protein is less than the degradation rate of said engineered protein of interest in said plant cell or a plant cell of the same species.

2. A method for decreasing the degradation rate of an engineered protein of interest in a plant cell comprising
constructing a vector comprising a nucleic acid fragment from position 1 to position 4831 of SEQ ID NO:3 fused to a nucleotide sequence encoding said protein of interest, said vector expressible in said plant cell; and
introducing and expressing said vector in said plant cell to form a fused protein;
wherein the degradation rate of said fused protein is less than the degradation rate of said engineered protein of interest in said plant cell or a plant cell of the same species.

3. A method for increasing the degradation rate of an engineered protein of interest in a plant cell comprising
constructing a vector comprising a nucleic acid fragment from position 1 to position 3348 of SEQ ID NO:5 fused to a nucleotide sequence encoding said protein of interest, said vector expressible in said plant cell; and
introducing and expressing said vector in said plant cell to form a fused protein;
wherein the degradation rate of said fused protein is less than the degradation rate of said engineered protein of interest in said plant cell or a plant cell of the same species.

4. The method of claim 3, wherein nucleotides at positions 1096-1098 of SEQ ID NO:5 encode alanine or tyrosine.

5. A method for increasing the degradation rate of an engineered protein of interest in a plant cell comprising
constructing a vector comprising a nucleic acid fragment from position 1 to position 4831 of SEQ ID NO:7 fused to a nucleotide sequence encoding said protein of interest, said vector expressible in said plant cell; and
introducing and expressing said vector in said plant cell to form a fused protein;
wherein the degradation rate of said fused protein is less than the degradation rate of said engineered protein of interest in said plant cell or a plant cell of the same species.

6. The method of claim 5, wherein nucleotides at positions 1096-1098 of SEQ ID NO:7 encode alanine or tyrosine.

7. The method of claim 1, 2, 3, 4, 5 or 6, wherein said vector is integrated into the genome of said plant cell.

8. A plant cell transformed according to a method comprising
constructing a vector comprising a nucleic acid fragment fused to a nucleotide sequence encoding said protein of interest, said nucleic acid fragment selected from the group consisting of from position 1 to position 3348 of SEQ ID NO:1, from position 1 to position 4831 of SEQ ID NO:3, from position 1 to position 3348 of SEQ ID NO:5, and from position 1 to position 4831 of SEQ ID NO:7, and said vector expressible in said plant cell; and
introducing and expressing said vector in said plant cell to form a fused protein;
wherein the degradation rate of said fused protein is less than the degradation rate of said engineered protein of interest in said plant cell or a plant cell of the same species.

9. A plant generated from the plant cell transformed according to a method comprising
constructing a vector comprising a nucleic acid fragment fused to a nucleotide sequence encoding said protein of interest, said nucleic acid fragment selected from the group consisting of from position 1 to position 3348 of SEQ ID NO:1, from position 1 to position 4831 of SEQ ID NO:3, from position 1 to position 3348 of SEQ ID NO:5, and from position 1 to position 4831 of SEQ ID NO:7, and said vector expressible in said plant cell; and
introducing and expressing said vector in said plant cell to form a fused protein;
wherein the degradation rate of said fused protein is less than the degradation rate of said engineered protein of interest in said plant cell or a plant cell of the same species.

10. A purified nucleic acid comprising a nucleic acid fragment from position 1 to position 3348 of SEQ ID NO:1 fused to a DNA sequence encoding a protein of interest.

11. The purified nucleic acid of claim 10, wherein expression of said purified nucleic acid in a plant cell results in a fusion protein having increased stability when compared to the stability of said protein of interest engineered without fusion to a nucleic acid fragment from position 1 to position 3348 of SEQ ID NO:1 expressed in a plant cell of the same species.

12. A purified nucleic acid comprising a nucleic acid fragment from position 1 to position 4831 of SEQ ID NO:3 fused to a DNA sequence encoding a protein of interest.

13. The purified nucleic acid of claim 12, wherein expression of said purified nucleic acid in a plant cell results in a fusion protein having increased stability when compared to the stability of said protein of interest engineered without fusion to a nucleic acid fragment from position 1 to position 4831 of SEQ ID NO:3 expressed in a plant cell of the same species.

14. A purified nucleic acid comprising a nucleic acid fragment from position 1 to position 3348 of SEQ ID NO:5 fused to a DNA sequence encoding a protein of interest.

15. The purified nucleic acid of claim 14, wherein expression of said purified nucleic acid in a plant cell results in a fusion protein having increased stability when compared to the stability of said protein of interest engineered without fusion to a nucleic acid fragment from position 1 to position 3348 of SEQ ID NO:5 expressed in a plant cell of the same species.

16. The purified nucleic acid of claim 14, wherein expression of said purified nucleic acid in a plant cell results in a fusion protein having decreased stability when compared to the stability of said protein of interest engineered without fusion to said nucleic acid fragment from position 1 to position 3348 of SEQ ID NO:5 expressed in a plant cell of the same species.

17. The purified nucleic acid of claim 14 or 16, wherein nucleotides at positions 1096-1098 of SEQ ID NO:5 encode alanine or tyrosine.

18. A purified nucleic acid comprising a nucleic acid fragment from position 1 to position 4831 of SEQ ID NO:7 fused to a DNA sequence encoding a protein of interest.

19. The purified nucleic acid of claim 18, wherein expression of said purified nucleic acid in a plant cell results in a fusion protein having increased stability when compared to the stability of said protein of interest engineered without fusion to a nucleic acid fragment from position 1 to position 4831 of SEQ ID NO:7 expressed in a plant cell of the same species.

20. The purified nucleic acid of claim 18, wherein expression of said purified nucleic acid in a plant cell results in a fusion protein having decreased stability when compared to the stability of said protein of interest engineered without fusion to said nucleic acid fragment from position 1 to position 4831 of SEQ ID NO:7 expressed in a plant cell of the same species.

21. The purified nucleic acid of claim 18 or 20, wherein nucleotides at positions 1096-1098 of SEQ ID NO:7 encode alanine or tyrosine.

22. A fusion protein comprising SEQ ID NO:2 fused to an amino acid sequence of interest.

23. The fusion protein of claim 22, wherein said fusion protein has increased stability in a plant cell compared to said amino acid sequence of interest in a plant cell of the same species.

24. A fusion protein comprising SEQ ID NO:4 fused to an amino acid sequence of interest.

25. The fusion protein of claim 24, wherein said fusion protein has increased stability in a plant cell compared to said amino acid sequence of interest in a plant cell of the same species.

26. A fusion protein comprising SEQ ID NO:6 fused to an amino acid sequence of interest.

27. The fusion protein of claim 26, wherein said fusion protein has increased stability in a plant cell compared to said amino acid sequence of interest in a plant cell of the same species.

28. The fusion protein of claim 26, wherein said fusion protein has decreased stability in a plant cell compared to said amino acid sequence of interest in a plant cell of the same species.

29. The fusion protein of claim 26 or 28, wherein the amino acid at position 366 of SEQ ID NO:6 is alanine or tyrosine.

30. A fusion protein comprising SEQ ID NO:8 fused to an amino acid sequence of interest.

31. The fusion protein of claim 30, wherein said fusion protein has increased stability in a plant cell compared to said amino acid sequence of interest in a plant cell of the same species.

32. The fusion protein of claim 30, wherein said fusion protein has decreased stability in a plant cell compared to said amino acid sequence of interest in a plant cell of the same species.

33. The fusion protein of claim 30 or 32, wherein the amino acid at position 366 of SEQ ID NO:8 is alanine or tyrosine.

34. A vector purified nucleic acid encoding a fusion protein comprising SEQ ID NO:2 fused to an amino acid sequence of interest.

35. A vector comprising a purified nucleic acid encoding a fusion protein comprising SEQ ID NO:2 fused to an amino acid sequence of interest.

36. A plant cell transformed by a vector comprising a purified nucleic acid, said purified nucleic acid selected from the group consisting of a purified nucleic acid encoding a fusion protein comprising SEQ ID NO:2 fused to an amino acid sequence of interest, a purified nucleic acid encoding a fusion protein comprising SEQ ID NO:4 fused to an amino acid sequence of interest; a purified nucleic acid encoding a fusion protein comprising SEQ ID NO:6 fused to an amino acid sequence of interest; and a purified nucleic acid encoding a fusion protein comprising SEQ ID NO:8 fused to an amino acid sequence of interest.

37. A plant generated from a plant cell transformed by a vector comprising a purified nucleic acid, said purified nucleic acid selected from the group consisting of a purified nucleic acid encoding a fusion protein comprising SEQ ID NO:2 fused to an amino acid sequence of interest, a purified nucleic acid encoding a fusion protein comprising SEQ ID NO:4 fused to an amino acid sequence of interest; a purified nucleic acid encoding a fusion protein comprising SEQ ID NO:6 fused to an amino acid sequence of interest; and a purified nucleic acid encoding a fusion protein comprising SEQ ID NO:8 fused to an amino acid sequence of interest.

38. A method for decreasing the degradation rate of an engineered protein of interest in a plant cell comprising
constructing a vector comprising a nucleic acid sequence that encodes a membrane binding protein from the Sindbis-like plant virus family fused to a nucleotide sequence encoding said protein of interest, said vector expressible in a plant cell; and
introducing and expressing said vector in said plant cell to form a fused protein;
wherein the degradation rate of said fused protein is less than the degradation rate of said engineered protein of interest in said plant cell or a plant cell of the same species.

39. The method of claim 38, wherein said membrane binding protein from the Sindbis-like plant virus family contains a “WFP” motif as depicted at amino acid position 365-367 of SEQ ID NO:2.

40. A method for increasing the degradation rate of an engineered protein of interest in a plant cell comprising
constructing a vector comprising a nucleic acid sequence that encodes a membrane binding protein from the Sindbis-like plant virus family fused to a nucleotide sequence encoding said protein of interest, said vector expressible in a plant cell; and
introducing and expressing said vector in said plant cell to form a fused protein;
wherein the degradation rate of said fused protein is less than the degradation rate of said engineered protein of interest in said plant cell or a plant cell of the same species.

41. The method of claim 40, wherein said membrane binding protein from the Sindbis-like plant virus family contains a mutation in the “WFP” motif as depicted at amino acid position 365-367 of SEQ ID NO:2.

42. The method of claim 38 or 40, wherein said vector is integrated into the genome of said plant cell.

43. The method of claim 38, 39, 40 or 41, wherein the Sindbis-like plant virus is selected from the group consisting of alfalfa mosaic virus, brome mosaic virus, citrus leaf rugose virus, cucumber mosaic virus, sunn-hemp mosaic virus, tobacco mosaic virus, tobacco rattle virus, and turnip vein clearing virus.

44. A plant cell transformed according to a method comprising
constructing a vector comprising a nucleic acid sequence that encodes a membrane binding protein from the Sindbis-like plant virus family fused to a nucleotide sequence encoding said protein of interest, said vector expressible in a plant cell; and
introducing and expressing said vector in said plant cell to form a fused protein;
wherein the degradation rate of said fused protein is less than the degradation rate of said engineered protein of interest in said plant cell or a plant cell of the same species.

45. A plant generated from a plant cell transformed according to a method comprising
constructing a vector comprising a nucleic acid sequence that encodes a membrane binding protein from the Sindbis-like plant virus family fused to a nucleotide sequence encoding said protein of interest, said vector expressible in a plant cell; and
introducing and expressing said vector in said plant cell to form a fused protein;
wherein the degradation rate of said fused protein is less than the degradation rate of said engineered protein of interest in said plant cell or a plant cell of the same species.

46. A purified nucleic acid comprising a nucleic acid fragment encoding a membrane binding protein from the Sindbis-like plant virus fused to a DNA sequence encoding a protein of interest.

47. A purified nucleic acid comprising a nucleic acid fragment encoding a membrane binding protein from the Sindbis-like plant virus containing a mutation in the “WFP” motif as depicted at amino acid position 365-367 of SEQ ID NO:2 fused to a DNA sequence encoding a protein of interest.

48. The purified nucleic acid of claim 46 or 47, wherein the Sindbis-like plant virus is selected from the group consisting of alfalfa mosaic virus, brome mosaic virus, citrus leaf rugose virus, cucumber mosaic virus, sunn-hemp mosaic virus, tobacco mosaic virus, tobacco rattle virus, and turnip vein clearing virus.

49. A fusion protein comprising a membrane binding protein from the Sindbis-like plant virus family fused to an amino acid sequence of interest.

50. A fusion protein comprising a membrane binding protein from the Sindbis-like plant virus family containing a mutation in the “WFP” motif as depicted at amino acid position 365-367 of SEQ ID NO:2 fused to an amino acid sequence of interest.

51. The fusion protein of claim 49 or 50, wherein said fusion protein has increased stability in a plant cell compared to said amino acid sequence of interest in a plant cell of the same species.

52. The fusion protein of claim 49 or 50, wherein said fusion protein has decreased stability in a plant cell compared to said amino acid sequence of interest in a plant cell of the same species.

53. The fusion protein of claim 49 or 50, wherein the Sindbis-like plant virus is selected from the group consisting of alfalfa mosaic virus, brome mosaic virus, citrus leaf rugose virus, cucumber mosaic virus, sunn-hemp mosaic virus, tobacco mosaic virus, tobacco rattle virus, and turnip vein clearing virus.

54. A nucleic acid fragment encoding a fusion protein comprising a membrane binding protein from the Sindbis-like plant virus family fused to an amino acid sequence of interest.

55. A vector comprising a nucleic acid fragment encoding a fusion protein comprising a membrane binding protein from the Sindbis-like plant virus family fused to an amino acid sequence of interest.

56. A plant cell transformed with a vector comprising a nucleic acid fragment encoding a fusion protein comprising a membrane binding protein from the Sindbis-like plant virus family fused to an amino acid sequence of interest.

57. A plant generated from a plant cell transformed with a vector comprising a nucleic acid fragment encoding a fusion protein comprising a membrane binding protein from the Sindbis-like plant virus family fused to an amino acid sequence of interest.

58. The plant cell of claim 8, wherein nucleotides at positions 1096-1098 of SEQ ID NO:5 encode alanine or tyrosine.

59. The plant cell of claim 8, wherein nucleotides at positions 1096-1098 of SEQ ID NO:7 encode alanine or tyrosine.

60. The plant cell of claim 8, 58 or 59, wherein said vector is integrated into the genome of said plant cell.

61. The plant of claim 9, wherein nucleotides at positions 1096-1098 of SEQ ID NO:5 encode alanine or tyrosine.

62. The plant of claim 9, wherein nucleotides at positions 1096-1098 of SEQ ID NO:7 encode alanine or tyrosine.

63. The plant of claim 9, 61 or 62, wherein said vector is integrated into the genome of said plant cell.

64. The vector purified nucleic acid of claim 34, wherein said fusion protein has increased stability in a plant cell compared to said amino acid sequence of interest in a plant cell of the same species.

65. A vector purified nucleic acid encoding a fusion protein comprising SEQ ID NO:4 fused to an amino acid sequence of interest.

66. The vector purified nucleic acid of claim 65, wherein said fusion protein has increased stability in a plant cell compared to said amino acid sequence of interest in a plant cell of the same species.

67. A vector purified nucleic acid encoding a fusion protein comprising SEQ ID NO:6 fused to an amino acid sequence of interest.

68. The vector purified nucleic acid of claim 67, wherein said fusion protein has increased stability in a plant cell compared to said amino acid sequence of interest in a plant cell of the same species.

69. The vector purified nucleic acid of claim 67, wherein said fusion protein has decreased stability in a plant cell compared to said amino acid sequence of interest in a plant cell of the same species.

70. The vector purified nucleic acid of claim 67 or 69, wherein the amino acid at position 366 of SEQ ID NO:6 is alanine or tyrosine.

71. A vector purified nucleic acid encoding a fusion protein comprising SEQ ID NO:8 fused to an amino acid sequence of interest.

72. The vector purified nucleic acid of claim 71, wherein said fusion protein has increased stability in a plant cell compared to said amino acid sequence of interest in a plant cell of the same species.

73. The vector purified nucleic acid of claim 71, wherein said fusion protein has decreased stability in a plant cell compared to said amino acid sequence of interest in a plant cell of the same species.

74. The vector purified nucleic acid of claim 71 or 73, wherein the amino acid at position 366 of SEQ ID NO:8 is alanine or tyrosine.

75. The vector of claim 35, wherein said fusion protein has increased stability in a plant cell compared to said amino acid sequence of interest in a plant cell of the same species.

76. A vector comprising a purified nucleic acid encoding a fusion protein comprising SEQ ID NO:4 fused to an amino acid sequence of interest.

77. The vector of claim 76, wherein said fusion protein has increased stability in a plant cell compared to said amino acid sequence of interest in a plant cell of the same species.

78. A vector comprising a purified nucleic acid encoding a fusion protein comprising SEQ ID NO:6 fused to an amino acid sequence of interest.

79. The vector of claim 78, wherein said fusion protein has increased stability in a plant cell compared to said amino acid sequence of interest in a plant cell of the same species.

80. The vector of claim 78, wherein said fusion protein has decreased stability in a plant cell compared to said amino acid sequence of interest in a plant cell of the same species.

81. The vector of claim 78 or 80, wherein the amino acid at position 366 of SEQ ID NO:6 is alanine or tyrosine.

82. A vector comprising a purified nucleic acid encoding a fusion protein comprising SEQ ID NO:8 fused to an amino acid sequence of interest.

83. The vector of claim 82, wherein said fusion protein has increased stability in a plant cell compared to said amino acid sequence of interest in a plant cell of the same species.

84. The vector of claim 82, wherein said fusion protein has decreased stability in a plant cell compared to said amino acid sequence of interest in a plant cell of the same species.

85. The vector of claim 82 or 84, wherein the amino acid at position 366 of SEQ ID NO:8 is alanine or tyrosine.

86. The plant cell of claim 36, wherein said fusion protein has increased stability in a plant cell compared to said amino acid sequence of interest in a plant cell of the same species.

87. The plant cell of claim 36, wherein said fusion protein has decreased stability in a plant cell compared to said amino acid sequence of interest in a plant cell of the same species.

88. The plant cell of claim 36, wherein the amino acid at position 366 of SEQ ID NO:6 is alanine or tyrosine.

89. The plant cell of claim 36, wherein the amino acid at position 366 of SEQ ID NO:8 is alanine or tyrosine.

90. The plant of claim 37, wherein said fusion protein has increased stability in a plant cell compared to said amino acid sequence of interest in a plant cell of the same species.

91. The plant of claim 37, wherein said fusion protein has decreased stability in a plant cell compared to said amino acid sequence of interest in a plant cell of the same species.

92. The plant of claim 37, wherein the amino acid at position 366 of SEQ ID NO:6 is alanine or tyrosine.

93. The plant of claim 36, wherein the amino acid at position 366 of SEQ ID NO:8 is alanine or tyrosine.

94. The plant cell of claim 44, wherein said membrane binding protein from the Sindbis-like plant virus family contains a mutation in the “WFP” motif as depicted at amino acid position 365-367 of SEQ ID NO:2.

95. The plant cell of claim 44, wherein said vector is integrated into the genome of said plant cell.

96. The plant cell of claim 94, wherein said vector is integrated into the genome of said plant cell.

97. The plant cell of claim 44, 94, 95 or 96, wherein the Sindbis-like plant virus is selected from the group consisting of alfalfa mosaic virus, brome mosaic virus, citrus leaf rugose virus, cucumber mosaic virus, sunn-hemp mosaic virus, tobacco mosaic virus, tobacco rattle virus, and turnip vein clearing virus.

98. The plant of claim 45, wherein said membrane binding protein from the Sindbis-like plant virus family contains a mutation in the “WFP” motif as depicted at amino acid position 365-367 of SEQ ID NO:2.

99. The plant of claim 45, wherein said vector is integrated into the genome of said plant cell.

100. The plant of claim 98, wherein said vector is integrated into the genome of said plant cell.

101. The plant of claim 45, 98, 99 or 100, wherein the Sindbis-like plant virus is selected from the group consisting of alfalfa mosaic virus, brome mosaic virus, citrus leaf rugose virus, cucumber mosaic virus, sunn-hemp mosaic virus, tobacco mosaic virus, tobacco rattle virus, and turnip vein clearing virus.

102. The fusion protein of claim 51, wherein the Sindbis-like plant virus is selected from the group consisting of alfalfa mosaic virus, brome mosaic virus, citrus leaf rugose virus, cucumber mosaic virus, sunn-hemp mosaic virus, tobacco mosaic virus, tobacco rattle virus, and turnip vein clearing virus.

103. The fusion protein of claim 52, wherein the Sindbis-like plant virus is selected from the group consisting of alfalfa mosaic virus, brome mosaic virus, citrus leaf rugose virus, cucumber mosaic virus, sunn-hemp mosaic virus, tobacco mosaic virus, tobacco rattle virus, and turnip vein clearing virus.

104. A nucleic acid fragment encoding a fusion protein comprising a membrane binding protein from the Sindbis-like plant virus family containing a mutation in the “WFP” motif as depicted at amino acid position 365-367 of SEQ ID NO:2 fused to an amino acid sequence of interest.

105. The nucleic acid fragment of claim 54 or 104, wherein the Sindbis-like plant virus is selected from the group consisting of alfalfa mosaic virus, brome mosaic virus, citrus leaf rugose virus, cucumber mosaic virus, sunn-hemp mosaic virus, tobacco mosaic virus, tobacco rattle virus, and turnip vein clearing virus.

106. A vector comprising a nucleic acid fragment encoding a fusion protein comprising a membrane binding protein from the Sindbis-like plant virus family containing a mutation in the “WFP” motif as depicted at amino acid position 365-367 of SEQ ID NO:2 fused to an amino acid sequence of interest.

107. The vector of claim 55 or 106, wherein the Sindbis-like plant virus is selected from the group consisting of alfalfa mosaic virus, brome mosaic virus, citrus leaf rugose virus, cucumber mosaic virus, sunn-hemp mosaic virus, tobacco mosaic virus, tobacco rattle virus, and turnip vein clearing virus.

108. A plant cell transformed with a vector comprising a nucleic acid fragment encoding a fusion protein comprising a membrane binding protein from the Sindbis-like plant virus family containing a mutation in the “WFP” motif as depicted at amino acid position 365-367 of SEQ ID NO:2 fused to an amino acid sequence of interest.

109. The plant cell of claim 56 or 108, wherein the Sindbis-like plant virus is selected from the group consisting of alfalfa mosaic virus, brome mosaic virus, citrus leaf rugose virus, cucumber mosaic virus, sunn-hemp mosaic virus, tobacco mosaic virus, tobacco rattle virus, and turnip vein clearing virus.

110. A plant generated from a plant cell transformed with a vector comprising a nucleic acid fragment encoding a fusion protein comprising a membrane binding protein from the Sindbis-like plant virus family containing a mutation in the “WFP” motif as depicted at amino acid position 365-367 of SEQ ID NO:2 fused to an amino acid sequence of interest.

111. The plant cell of claim 57 or 110, wherein the Sindbis-like plant virus is selected from the group consisting of alfalfa mosaic virus, brome mosaic virus, citrus leaf rugose virus, cucumber mosaic virus, sunn-hemp mosaic virus, tobacco mosaic virus, tobacco rattle virus, and turnip vein clearing virus.
